SEQUENCE LISTING (1 sequence)

SEQ ID NO: 1 - protein, 13 aa, Homo sapiens
SYSMEHFRWGKPV

(19) United States
(12) Patent Application Publication
(10) Pub. No.: US 2003/0219400 A1
(43) Pub. Date: Nov. 27, 2003

(54) Methods for treating or inhibiting neurotoxin-mediated syndromes

(76) Inventors: Ritchie Shoemaker, Pocomoke, MD (US); H. Kenneth Hudnell, Pocomoke, MD (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/364,613

(22) Filed: Feb. 10, 2003

Related U.S. Application Data
(60) Provisional application No. 60/356,443, filed on Feb. 13, 2002.
(60) Provisional application No. 60/356,539, filed on Feb. 13, 2002.

(51) Int. Cl. A61K 38/23; A61K 31/785

(52) U.S. Cl. 424/78.1; 514/12

(57) ABSTRACT

The present invention discloses methods of treating or inhibiting one or more of sick building syndrome (SBS), post-Lyme Disease Syndrome (PLDS), and chronic fatigue syndrome (CFS) by administering to a patient in need thereof an amount effective of cholestyramine and/or α-melanocyte stimulating hormone to treat or inhibit one or more of these syndromes.

### CROSS REFERENCE

This application claims priority to U.S. provisional application serial No. 60/356,443, filed Feb. 13, 2002, and U.S. provisional application serial No. 60/356,539, filed Feb. 13, 2002.

### FIELD OF INVENTION

The present invention relates to the field of medicine, chronic illnesses, and pharmaceuticals.

### BACKGROUND OF THE INVENTION

Human illness associated with neurotoxin-forming environmental microbial organisms has been reported. For instance, the human illness designated as possible estuarine-associated syndrome (PEAS) has been linked to exposure to estuaries inhabited by toxin-forming dinoflagellate. Humans may be exposed through direct contact with estuarine water or by inhalation of aerosolized or volatilized toxin(s) (Shoemaker, R C, (1997) Md Med J. 46:521; Shoemaker, R C et al. Environ Health Perspect (2001) 109:539) The acute and chronic symptoms associated with the disease include cough, secretory diarrhea, headache, fatigue, memory impairment, rash, difficulty in concentrating, light sensitivity, burning skin upon water contact, muscle ache, upper airway obstruction, shortness of breath, confusion, red or tearing eyes, weakness and vertigo. In the absence of a serological test, and the identification of a specific dinoflagellate neurotoxin, the diagnosis of PEAS and the link to neurotoxin relies on a neurotoxicological test, visual contrast sensitivity (VCS). A toxin-trapping agent, cholestyramine (CSM), has been used in patients diagnosed with PEAS that have a deficit in VCS (Shoemaker, R C et al. Environ Health Perspect (2001) 109:539).

Sick building syndrome (SBS), or indoor air acquired illness, has been reported in the United States since 1970s. It is generally believed that inadequate dilution of irritants and noxious matter re-circulating in the air are the risk factors, but a clear etiology is lacking. The symptoms of SBS include chronic sinus congestion, cough, shortness of breath and wheezing may suggest allergies. Other patients with presumed SBS have irritant symptoms such as rashes, headaches, red eyes and tearing. Additional symptoms of sensitivity to bright light, memory impairment, difficulty in assimilating new knowledge, tingling in extremities in a non-anatomic distribution, vertigo, metallic taste, depression, cranial neuropathies, muscle aches, cramps, chronic small joint stiffness and migratory joint pain, profound fatigue, chronic abdominal pain and a sensation of weakness all can be found in SBS patients, which are not cleared by conventional SBS treatments. SBS may be a multifactorial condition, involving in some cases volatile organic compounds, carbon monoxide or carbon dioxide, pesticides, biologic agents, temperature and humidity, lighting, and neuropsychological status. Conventional SBS treatments include antibiotics, asthma or allergy medications or removal of the patient from the affected indoor air environment.

Since the first description of Lyme disease there have always been subsets of patients identified with persistent symptoms, refractory to antibiotics, referred to as the Post-Lyme Disease Syndrome (PLDS). PLDS patients often experience chronic fatigue, weak, multiple cognitive impairments, headache, sensitivity to bright light, redness, tearing, blurred vision, chronic sinus congestion, cough, shortness of breath, muscle aches, cramps, chronic non-specific abdominal pain. Conventional management of these patients has been ineffective, with many patients taking prolonged courses of antibiotics or antidepressants, for example, with mixed results.

Chronic fatigue syndrome (CFS) is the current name for a disorder characterized by debilitating fatigue and several associated physical, constitutional, and neuropsychological complaints, without any other confounding illness. The symptoms of CFS include fatigue, difficulty concentrating, impairment in short term memory, headache, sore throat, tender lymph node, muscle aches, joint aches, feverishness, difficulty sleeping, psychiatric problems, allergies, abdominal cramps, weight loss, rash, rapid pulse, weight gain, chest pain, and night sweat, and post-exertional malaise lasting more than 24 hours. Despite a widespread research into the health problems associated with chronic fatigue syndrome, including a possible link to persistent viral infections, neither a clearly recognized etiologic factor nor a routinely effective therapeutic modality has been identified.

It has been recognized as part of the present invention that there is a marked similarity between the persistent symptoms seen in SBS, PLDS, and CFS patients and the persistent symptoms experienced by patients with exposure to known environmental toxins. Previous work in PEAS patients whose chronic symptoms were acquired following exposure to various neurotoxin-forming species, including dinoflagellates, fungi and blue green algae, showed that these patients demonstrated a reproducible pattern of deficits in visual contrast sensitivity (VCS). The VCS deficits and the chronic symptoms in those patients with known neurotoxin mediated illnesses abated following treatment with cholestyramine (CSM) (Shoemaker, R C et al. Environ Health Perspect (2001) 109:539). However, no generally effective methods for treating or inhibiting the development of sick building syndrome, post-Lyme disease syndrome, or chronic fatigue syndrome are currently available, and thus, it would be highly beneficial to the art if such methods were developed.

### SUMMARY OF THE INVENTION

The present invention discloses methods of treating or inhibiting one or more of sick building syndrome (SBS), post-Lyme Disease Syndrome (PLDS), and chronic fatigue syndrome (CFS) by administering to a patient in need thereof an amount effective of cholestyramine and/or α-melanocyte stimulating hormone to treat or inhibit one or more of these syndromes. In a preferred embodiment, the patient treated suffers from a deficit in visual contrast sensitivity.

In a further aspect, the present invention provides a diagnostic method for identifying patients with SBS, PLDS, and/or CFS that would best benefit by treatment with cholestyramine and/or α-melanocyte stimulating hormone, comprising identifying those SBS, PLDS, and/or CFS patients that have a deficit in visual contrast sensitivity, and treating those patients with the deficit in visual contrast sensitivity with cholestyramine and/or α-melanocyte stimulating hormone.

### DETAILED DESCRIPTION OF THE INVENTION

In one aspect, the present invention discloses methods of treating or inhibiting one or more of sick building syndrome (SBS), post-Lyme disease Syndrome (PLDS), and chronic fatigue syndrome (CFS) by administering to a patient in need thereof an amount effective of cholestyramine to treat or inhibit one or more of these syndromes.

As used herein, “sick building syndrome” (SBS)(sometimes referred to as indoor air acquired illness; tight-building syndrome, and non-specific building-related illness), is a chronic illness for which a clear etiology is lacking, but which occur most often in sealed buildings with a centrally controlled mechanical ventilation and in buildings with water intrusion and evidence of fungal growth, also called mold, as noted by visible growth or musty smells. Risk factors are thought to include fungal contamination and inadequate dilution of irritants and noxious matter re-circulating in the air in such buildings.

As used herein, “post-Lyme disease syndrome” (PLDS) refers to persistent symptoms, refractory to standard antibiotic therapy, following acquisition of Lyme disease (which is itself a tick-transmitted spirochete illness).

As used herein, “chronic fatigue syndrome” (CFS) refers to a disorder characterized by debilitating fatigue and several associated physical, constitutional, and neuropsychological complaints, without any other confounding illness.

The chronic symptoms of SBS, PLDS, and CFS can include symptoms from four of eight organ systems, including fatigue, weakness, muscle aches, muscle cramps, headaches, unusual pains (sharp stabbing pains, “electical, lightning bolt” pains), sensitivity to bright light, red eyes, tearing, blurred vision, chronic sinus congestion, shortness of breath, cough, abdominal pain, secretory diarrhea, “irritable bowel disease,” joint pain, morning stiffness of small joints, sensitivity to light touch, cognitive dysfunction including short term memory impairment, confusion, reduced ability to concentrate, disorientation, delayed assimilation of new knowledge, difficulty with word finding; numbness, tingling, metallic taste, vertigo, mood swings, appetite swings, sweats (especially at night), difficulty regulating body temperature, excessive thirst, frequent urination and increased sensitivity to static electrical shocks.

Cholestyramine (also referred to herein as CSM) is a hydrophilic polyacrylic quaternary ammonium anion exchange resin, which is known to be effective in reducing blood cholesterol levels. Cholestyramine, and various compositions including cholestyramine, are described, for example, in British Pat Nos. 929,391 and 1,286,949; and U.S. Pat. Nos. 3,383,281; 3,308,020; 3,769,399; 3,846,541; 3,974,272; 4,172,120; 4,252,790; 4,340,585; 4,814,354; 4,874,744; 4,895,723; 5,695,749; and 6,066,336. Cholestyramine is commercially available from Novopharm, USA Inc. (Questran® Light), Upsher-Smith (PREVALITE®), and Apothecon.

As used herein, “cholestyramine” includes any such composition comprising cholestyramine, or pharmaceutically acceptable salts thereof.

As used herein “treating” the disorder(s) means decreasing or eradicating one or more of the symptoms associated with the disorder as described above, as well as to eliminate the disorder in the patient entirely. “Treating” may also encompass improvement in hypothalamic hormone measures, such as increased MSH levels and decreased leptin levels (as discussed below). In a preferred embodiment, treating includes decreasing or eradicating the deficit in visual contrast sensitivity (VCS).

As used herein “inhibiting” the disorder(s) refers to limiting the re-occurrence of symptoms of SBS, PLDS, and/or CFS in a patient who previously suffered from SBS, PLDS, and/or CFS. Such re-occurrence can result in reacquisition of symptoms that respond to repeat CSM therapy. A prophylactic administration of CSM in such patients is thus desirable if the possibility of re-exposure is a concern.

In a preferred embodiment, CSM is administered to a patient that is refractory to standard therapies for the disorder being treated. Such standard treatments are many and include, but are not limited to:

SBS: antibiotics; asthma or allergy medications and/or removal from the indoor environment;

PLDS: antibiotics and/or antidepressants to treat symptoms; and

CFS: Non-steroidal anti-inflammatory drugs, antihistamines, decongestants, anti-depressants; all to treat symptoms of the disorder.

In a further preferred embodiment, CSM is administered to a patient with one or more of SBS, PLDS, and CFS, wherein the patient has been exposed to a toxin-forming organism selected from the group consisting of dinoflagellates, fungi, spirochetes, protozoa, cyanobacteria, and bacteria. As part of the invention, is has been discovered that there is a marked similarity between the persistent symptoms seen in SBS, PLDS, and CFS patients and the persistent symptoms experienced by PEAS patients with exposure to known environmental toxins, which are effectively treated with CSM.

As used herein the term “exposed to a toxin-forming organism” refers to any patient contact with the toxin, such as direct contact with the toxin-producing organism itself, or by contact with the toxin, produced by the microbial organism. Such contact can occur, for example, by ingestion, inhalation, or contact through skin or mucosal membrane. Such toxin-forming dinoflagellates include but are not limited to Pfiesteria, Ciguatera, and Chattonella. Such toxin-forming fungi include but are not limited to Stachybotrys, Penicillium, Aspergillus, Cladosporium, and Fusarium. Such toxin-forming spirochetes include but are not limited to Borrelia, Treponema, Leptospira, and Denticola. Such toxin-forming protozoa include, but are not limited to Babesia and Plasmodium. Such toxin-forming cyanobacteria include but are not limited to Microcystis, Anabaenopsis and Cylindrospermopsis. Such toxin-forming bacteria include but are not limited to Bacillus, Clostridia, and coagulase-negative Staphylococcus.

In a further preferred embodiment, the patient treated with CSM suffers from a deficit in visual contrast sensitivity (VCS). Previous work with patients whose chronic symptoms were acquired following exposure to various neurotoxin-forming species, including dinoflagellates, fungi and blue green algae, showed that these patients demonstrated a reproducible pattern of deficits in VCS. The VCS deficits and the chronic symptoms in those patients with PEAS abated following CSM therapy. (Shoemaker and Hudnell, Environmental Health Perspectives 109:539 (2001)). While not being limited by any mechanism, it is thought that the deficit in VCS in these patients may be due to hypoperfusion of the optic nerve head similar but not identical to deficit associated with the hypoperfusion due to glaucoma.

VCS is determined by a test that measures the patient's visual ability to separate black from gray from white, as described in Shoemaker and Hudnell, Environmental Health Perspectives 109:539 (2001) and Shoemaker, Environmental Health Perspectives 109:791 (2001). As opposed to visual acuity, a measure that reflects 100% contrast between black and white, VCS testing isolates the functioning of neurons of the optic nerve and optic radiation that enable a visual image to include an edge. Previous work has shown that deficits in VCS are associated with abnormalities in visually evoked potentials. Deficits in contrast may be permanent, resulting from ophthalmologic diseases, neurodegenerative diseases, and occupational exposure to known neurotoxic substances, including organic solvents, heavy metals and petroleum products. Deficits in contrast that are not durable have been demonstrated to be present in chronic illnesses such as PEAS.

Thus, in at least a subset of patients, the progress of disease treatment can be monitored by periodically testing the VCS of patients undergoing treatment, where an improvement in VCS score correlates with treatment efficacy.

Any method for measuring VCS can be used with the methods of the invention. In a preferred embodiment, VCS is tested using a standardized measure, FACT® (Stereo-Optical, Chicago, Ill., a Gerber Coburn company) as described in Shoemaker, Environmental Health Perspectives 109:791 (2001). Briefly, the VCS test is a contrast sensitivity test card (FACT® 101) containing a matrix (5×9) of circles filled with sinusoidal gratings (dark and light bars). Spatial frequency (1.5, 3, 6, 12, and 18 cycles/degree of visual arc) increase from top to bottom, and contrast decreases from left to right in steps of approximately 0.15 log units. The grating bars are oriented either vertically or tilted 15 degrees to the left or the right. As the investigator calls out each circle from left to right, row by row, subjects respond by saying either vertical, right, left, or blank. Subjects are encouraged to name an orientation if they had an indication that the bars could be seen. Subjects (primarily younger children) are asked to point in the direction to which the top of the grating was tilted if they felt any difficulty in verbalizing the orientation. The VCS score for each row (spatial frequency) is recorded as the contrast of the last test patch correctly identified on that row following verification by repeated testing of that patch and the subsequent patch. The procedure is repeated for each row in descending order. It is required that subjects have a visual acuity of 20:50 or better (Snellen Distance Equivalent Score) (corrected or uncorrected) in order to avoid confounding of the VCS results by excessive optical-refraction error. The units of analysis for the VCS test are the mean scores of the subject's two eyes at each spatial frequency.

Prior, well-controlled studies have shown that a mid frequency VCS deficit, greatest at 6 cycles per degree of visual arc, when associated with known toxin-forming organism exposure and the presence of 4 of 8 categories of symptoms, together with a lack of confounding exposures, is a statistically validated measure confirming the presence of a neurotoxin effect.

Alternatively (or in combination), VCS can be measured using the Heidelberg Retinal Flowmeter, which records the velocity of capillary blood flow in the retina, neural rim of the optic nerve and lamina cribosa, the deepest layer of the optic nerve. The neural rim flow rate is markedly diminished, but not capillary flow rates, in symptomatic patients with a deficit in visual contrast sensitivity.

While not being limited by any specific mechanism, it is possible that CSM alleviates the deficit in visual contrast sensitivity through an electrostatic interaction of its quaternary ammonium side chains with a molecular dipole (ion sink) created by the molecular structure of toxins associated with the disorders. The electrostatic interaction may sequester the toxins in the gut, preventing enterohepatic recirculation.

In another aspect, the invention provides methods for treating or inhibiting one or more neurotoxin-associated syndrome, comprising administering to a patient in need thereof an amount effective of cholestyramine to treat or inhibit the one or more neurotoxin-associated syndrome. In a preferred embodiment of this aspect, the neurotoxin-associated syndrome is selected from the group of disorders caused by exposure to one or more neurotoxin-forming organisms selected from the group consisting of dinoflagellates, fungi, spirochetes, protozoa, cyanobacteria, gram-positive bacteria, apicomplexans, and arachnids. In further preferred embodiments of this aspect of the invention, the toxin-forming dinoflagellate is one or more of Pfiesteria, Ciguatera, and Chattonella; the toxin-forming fungi are one or more of Stachybotrys, Penicillium, Aspergillus, Cladosporium, and Fusarium; the toxin-forming spirochetes are one or more of Borrelia, Treponema, Leptospira, and Denticola; the toxin-forming protozoa are Apicomplexans selected from one or both of Babesia, Sarcocystis, and Plasmodium; the toxin-forming cyanobacteria are one or more of Microcystis, Anabaenopsis and Cylindrospermopsis; the toxin-forming gram positive bacteria are one or more of Bacillus, Clostridia, and coagulase negative Staphylococcus; and the toxin-forming arachnid is the brown recluse spider.

As used herein the term “exposed to a toxin-forming organism” refers to any patient contact with the toxin, such as direct contact with the toxin-producing organism itself, or by contact with the toxin, produced by the microbial organism. Such contact can occur, for example, by ingestion, inhalation, or contact through skin or mucosal membrane.

Such neurotoxin-associated syndromes are expected to produce symptoms similar to those described above for SBS, PLDS, and CFS. Thus, treating and inhibiting these neurotoxin-associated disorders is defined as above.

We have found that some individuals exposed to the neurotoxin-forming organisms discussed above acquire only acute illness or are unaffected, while others develop the chronic disorders discussed herein. Thus, we suspected that susceptibility to chronic illness was conferred by particular patterns of genetic polymorphisms in class II human leukocyte antigen (HLA) alleles, given their role in antigen presentation to immune T cells.

Thus, 10 HLA alleles in 380 chronically ill patients with documented exposure potentials were assessed by standard PCR analysis. These analyses identified unique and disproportionate patterns of HLA alleles, relative to the entire patient population and to published frequencies in a large normal population. Statistically significant differences in allelic fingerprints were found, with various disorders described herein associated with the following HLA triplets:

Dinoflagellate-related illness: DRB1-4, DQ-8, and DRB4-53; or DRB1-4, DQ7 or DQ8, and DRB4-53

SBS (fungal exposure): DRB1-17, DQ-2, and DRB3-52A; subsequent studies demonstrated that the following HLA triplets were particularly associated with susceptibility to SBS: DRB1-7, DQ2, DRB4-53; DRB1-7, DQ9, DRB4-53; DRB1-13, DQ6, DRB3-52A/B/C; and DRB1-17, DQ2, DRB3-52A;

Nasal resident coagulase negative Staphylococcus-related illness: DRB1-11, DQ-7, and DRB3-52B;

PLDS: DRB1-15, DQ-6, DRB4-51; and DRB1-16, DQ-5, DRB4-51;

Susceptibility to neurotoxin-associated syndromes associated with exposure to coagulase negative Staphylococcus is associated with HLA DR genotype DRB1-11, DQ7, DRB3-52B; and

Susceptibility to CFS is associated with HLA DR genotype DRB1-4, DQ3, DRB4-53.

The specific allele patterns disclosed above accurately separated patients by diagnosis; each pattern was present in over 90% of the patients in the appropriate class, and in less than 5% of the patients in a different class. The few patients with illness suspected to involve two different types of exposure had allele patterns associated with risk from both types of exposure. In addition, several patients with histories of illness in more than two of the patient classes showed another allele pattern: DRB1-14, DQ-5, and DRB3-52B. Thus, these distinct genetic HLA patterns are risk factors for chronic illness as a result of exposure to particular classes of neurotoxin-forming organisms.

Thus, in another aspect, the present invention comprises methods for treating or inhibiting one or more neurotoxin-associated syndrome, including those discussed above, and further including PEAS, ciguatera, fibromyalgia, and irritable bowel syndrome, comprising administering to a patient suffering from one or more neurotoxin-associated syndrome, or who has recovered from one or more such neurotoxin-associated syndrome as described above and that possesses one of the HLA allelic genotypes described above an amount effective of cholestyramine to treat or inhibit the one or more neurotoxin-associated syndrome. Thus, in one embodiment, a patient with a neurotoxin-associated syndrome and with an HLA genotype that includes the triples DRB1-4, DQ8, DRB4-53, is treated with CSM according to the methods of the invention. Further such specific treatments will be apparent to one of skill in the art based on the teachings herein

HLA genotyping can be accomplished by commercially available methods available to one of skill in the art. (For example, Orchid BioScience Inc. (Princeton, N.J.); Applied BioSystems, CA) In a preferred embodiment, polymerase chain reaction (PCR) analysis is used for the genotyping. As used herein, HLA genotyping is a determination of the particular major histocompatibility complex (MHC) alleles expressed by an individual. PCR has been used to permit more complete typing of class II loci and has replaced both serology and secondary MLRs (Mixed Leukocyte Rections). The polymorphic residues of class II MHC molecules are largely located within exon 2 of both the α and β chains. The entire region of the gene can be amplified by PCR with primers that bind to conserved sequences within the 5′ and 3′ ends of these exons. (See, for example, U.S. Pat. Nos. 6,194,147 and 5,759,771)

In a subset of patients suffering from a neurotoxin-associated syndrome that are treated with CSM, therapy is ineffective or improvement is not sustained. While not being bound by a specific mechanism, toxins released from toxin-forming organisms, such as those discussed above, can activate excessive release of pro-inflammatory cytokines. Such toxins can also selectively activate cytokine receptors and downstream signaling pathways. Pro-inflammatory cytokines are made by fat cells in great quantities. The expression of these cytokines is controlled by a large family of cytokine nuclear receptors, which can be activated by a variety of compounds. When these overlapping, redundant systems of cytokine production are out balance, illness will result.

Disturbances in endocrine function, consistent with reduced production of corticotropin-releasing hormone in the hypothalamus, have been reported in controlled studies of CFS. The role of a hypothalamic hormone, alpha-melanocyte stimulating hormone (α-MSH) has not been investigated in SBS, PLDS, or CFS patients, despite the importance of MSH in downstream production of endorphins and melatonin, activation of MC4 receptors in the ventromedial nucleus of the hypothalamus, and marked anti-inflammatory peripheral activity as evidenced by down-regulation of macrophage release of pro-inflammatory cytokines, including tumor necrosis factor alpha (TNF) and interleukin-1-beta (IL1B) and possibly others, following intraventricular instillation of αMSH in experimental animals. αMSH is a tridecapeptide of sequence Ser-Tyr-Ser-Met-Glu-His-Phe-Arg-Trp-Gly-Lys-Pro-Val (SEQ ID NO:1).

We have seen that deficiencies in MSH are commonly seen in patients with elevated levels of pro-inflammatory cytokines associated with exposure to environmental sources of biotoxin production, including indoor toxin forming fungi leading to SBS. The pro-opiomelanocortoid pathway (POMC) generates MSH in the hypothalamus. Leptin is an agonist of cytokine receptors that initiate activity of the POMC. Serum leptin can be measured looking for evidence of reduced receptor activity for the leptin agonist, and thus an elevated level of leptin is a marker of impaired MSH production. The inability of the POMC pathway to make MSH is marked by refractory symptoms, such as obesity and leptin resistance.

In a subset of patients with neurotoxin-associated syndromes that are treated with CSM, binding neurotoxins out of the extravascular circulation with CSM can set off a “storm” of cytokine release, heralded by a rise of TNF mRNA. When patients suffer the effects of a rapidly rising TNF (TNF mRNA is a marker for subsequent TNF activity), whether from antibiotic treatment, CSM treatment, or some other cause, the patients can develop severe symptoms of the underlying disorder.

Thus, in another aspect, the present invention provides methods for treating or inhibiting one or more of SBS, PLDS and CFS, and/or one or more of the neurotoxin-associated syndrome discussed above, by administering to a patient in need thereof an amount effective of α-melanocyte stimulating hormone (αMSH) to treat or inhibit one or more of these syndromes. The αMSH can be administered alone, in addition to CSM, or can be administered subsequent to CSM treatment. In a preferred embodiment, the patient treated suffers from a deficit in visual contrast sensitivity, as discussed above. In a further preferred embodiment, the patient is refractory to standard treatments for the relevant disorder. In further preferred embodiments, the patient has one or more of the HLA genotypes that are linked to susceptibility to neurotoxin-associated syndromes, as discussed above.

In a further embodiment of this aspect of the invention, the patient to be treated with αMSH is determined to have one or more of a relative decrease in serum αMSH, an increase in plasma TNF or TNF mRNA, an increase in serum IL-1B or IL-1B mRNA relative to, or increased leptin or leptin mRNA levels relative to a healthy subject. In a preferred embodiment, the patient to be treated is identified as having a level of any of the foregoing 25% or more above laboratory standards for a normal subject.

In a further embodiment, the level of TNF, TNF mRNA, IL-1B, IL-1B mRNA, leptin, and/or leptin mRNA that was elevated relative to a healthy individual is normalized by the treatment with αMSH.

In further embodiments, markers used for analysis include matrix metalloprotease 9 (MMP9) and others described in the examples to follow.

Measurement of plasma or serum TNF, TNF mRNA, IL-1B, IL-1B mRNA, leptin, and/or leptin mRNA can be carried out by standard methods in the art, including but not limited to polymerase chain reaction techniques, gene array analysis, Western blot analysis, enzyme-linked immunofluorescent assays, and other methods known to one of skill in the art. See, for example, Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); and PCR Protocols: A Guide to Methods and Applications (Innis, et al. 1990. Academic Press, San Diego, Calif.).

The MSH polypeptide can be prepared from natural sources, recombinantly produced, or chemically synthesized. Preferably, the MSH is chemically synthesized. Synthetic polypeptides, prepared using the well known techniques of solid phase, liquid phase, or peptide condensation techniques, or any combination thereof, can include natural and unnatural amino acids. Amino acids used for peptide synthesis may be standard Boc (Nα-amino protected Nα-t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile Nα-amino protected 9-fluorenylmethoxycarbonyl (Fmoc) amino acids first described by Carpino and Han (1972, J. Org. Chem. 37:3403-3409). Both Fmoc and Boc Nα-amino protected amino acids can be obtained from Sigma, Cambridge Research Biochemical, or other chemical companies familiar to those skilled in the art. In addition, the polypeptides can be synthesized with other Nα-protecting groups that are familiar to those skilled in this art.

Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept. Protein Res. 35:161-214, or using automated synthesizers. The MSH polypeptides of the invention may comprise D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties. Synthetic amino acids include ornithine for lysine, and norleucine for leucine or isoleucine.

In addition, the MSH polypeptide can have peptidomimetic bonds, such as ester bonds, to prepare peptides with novel properties. For example, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo.

Activation of cytokine nuclear receptors in adipocytes may cause adipocytes to release pro-inflammatory cytokines, which are the main source of symptoms in PLDS. Extravascular organisms, such as the causative organism of Lyme disease, and their fat-soluble ionophore (cross from cell to cell) neurotoxins, can cause continuous activation of adipocyte cytokine receptors. Activated PPAR receptors, found essentially only in fat cells, serve to down-regulate the cytokine nuclear receptor effects. Thus, in another embodiment, combined use of a PPAR agonist with CSM can serve to alleviate potential CSM-mediated TNF-derived side effects of CSM treatment of SBS, PLDS, CFS, and/or neurotoxin-associated syndromes as disclosed herein. Any such PPAR agonist can be used. In a preferred embodiment, the PPAR agonist is a thiazolidinedione (TZD). In a more preferred embodiment, the TZD is selected from the group consisting of cioglitazone (U.S. Pat. Nos. 6,207,690; 5,814,647), rosiglitazone (Avandia) and pioglitazone (Actos).

In a further embodiment, CSM and/or αMSH can be used in combination with atovaquone (GlaxoSmithKline) to treat or inhibit SBS, PLDS, CFS, and/or neurotoxin-associated syndromes as described herein. In a preferred embodiment, the methods comprise administration of CSM and atovaquone in patients with co-infections from Lyme disease and Babesia, Sarcocystis and in Apicomplexan infections without coinfection with Lyme, refractory to standard antibiotic therapy.

The compounds to be administered according to the methods of the invention may be subjected to conventional pharmaceutical operations such as sterilization and/or may contain conventional adjuvants, such as preservatives, stabilizers, wetting agents, emulsifiers, buffers etc.

For administration, the compounds to be administered are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In practicing the methods of the invention, the amount or dosage range of the compounds to be administered, as well as the duration of such administration employed is one that effectively treats and/or inhibits SBS, PLDS, CFS and/or one or more of the neurotoxin-associated syndromes as described above, which can be determined by an attending physician based on a variety of factors, including but not limited to age, gender, weight, severity of symptoms, and overall health. Standard dosages of CSM that are used for other indications are useful with the methods of the invention. In a preferred embodiment, the patient takes between 1 and 15 grams of powder and 1-8 grams of resin; most optimally, 9 grams of powder and 4 grams of resin. These dosages are preferably administered 1-5 times per day, and most optimally 4 times per day.

A preferred dosage of αMSH is between 1 μg/kg/day and 50 μg/kg/day; more preferably between 2 μg/kg/day and 20 μg/kg/day, and most preferably between 5 μg/kg/day and 10 μg/kg/day.

A preferred dosage of rosiglitazone or pioglitazone is between 1 μg/kg and 100 μg/kg; more preferably between 10 μg/kg and 100 μg/kg, and most preferably between 50 μg/kg and 100 μg/kg, once or twice per day.

A preferred dosage of atovaquone is between 100 μg/kg and 20 mg/kg; more preferably between 1 mg/kg and 20 mg/kg, and most preferably between 5 mg/kg and 20 mg/kg, twice per day.

The compounds may be administered by any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. In preferred embodiments, CSM is administered orally. In a further preferred embodiment, αMSH is administered intra-nasally.

The compounds may be made up in a solid form (including granules, powders or suppositories) or in a liquid form (e.g., solutions, suspensions, or emulsions). The polypeptides of the invention may be applied in a variety of solutions. Suitable solutions for use in accordance with the invention are sterile, dissolve sufficient amounts of the compounds, and are not harmful for the proposed application.

In a further aspect, the present invention provides a diagnostic method for identifying patients with SBS, PLDS, and/or CFS that would best benefit by treatment with the methods of the invention, comprising identifying those SBS, PLDS, and/or CFS patients that have a deficit in visual contrast sensitivity, and treating those patients with the deficit in visual contrast sensitivity with the methods of the invention as described above.

In a still further aspect, the present invention provides a diagnostic method for identifying patients with SBS, PLDS, CFS, and/or a neurotoxin-associated syndrome as described above that would best benefit by treatment with the methods of the invention, comprising identifying those SBS, PLDS, and/or CFS patients that have one or more of the HLA genotypes disclosed above as being linked to susceptibility to a neurotoxin-associated disorder.

The present invention may be better understood with reference to the accompanying examples that are intended for purposes of illustration only and should not be construed to limit the scope of the invention, as defined by the claims appended hereto.

### EXAMPLES

### Example 1

### Sick Building Syndrome (SBS): Possible Association with Exposure to Mycotoxins from Indoor Air Fungi

The primary causative factor in a subset of SBS cases may be exposure to mycotoxins from indoor air fungi. A variety of fungal genera, including Stachybotrys, Aspergillus, Penicillium and Cladosporium, have been identified on interior cellulose materials following water intrusion. Many species have been shown to produce mycotoxins and release spores in the air. Mycotoxin exposure has been associated with effects on the nervous, digestive, respiratory, cutaneous, urinary, reproductive, immune and other systems.

Methods: As in other biotoxin-induced chronic human illnesses for which techniques to identify toxins in tissues are unavailable, SBS patient diagnosis was based on exposure potential, the presence of multiple system symptoms, and the absence of probable alternative causes of illness. Samples of fungi were observed growing in each building and analytically identified to assess exposure potential. Symptoms were systematically assessed in direct patient interviews. A test of visual pattern detection ability, visual contrast sensitivity (VCS), was administered to each SBS patient to assess its usefulness as an objective indicator of neurotoxicity in mycotoxicosis. Alternative explanations of illness were assessed with clinical and laboratory techniques, as well as with questions on medical history, potential for exposure to other toxins and life style. Cases were treated solely with an orally administered, non-absorbable polymer, cholestyramine (CSM), that binds salts from bile through anion exchange.

Results. One or more genera of toxin forming fungi were identified in each building that the patients either worked or lived in. All patients reported neurologic symptoms and symptoms involving at least three other systems. All cases showed depressed VCS in the presence of normal visual acuity, indicative of a neurologic effect. No probable alternative causes of illness were identified. Following 2 weeks of CSM therapy in the absence of re-exposure, all cases showed normal VCS and at least a 90% resolution of symptoms. Relapse occurred only with re-exposure and resolved with re-treatment with CSM.

Conclusions. Even in the absence of measures of airborne spore concentrations and mycotoxin levels in tissue, these results strongly support the hypothesis of mycotoxicosis in these SBS patients. Multiple system symptoms, the objective indication of a neurologic effect provided by VCS, relapse with re-exposure and successive recoveries following CSM therapy are consistent with this diagnosis. The CSM response in these chronically ill patients unresponsive to previous treatments has no known explanation other than enhancement of toxin elimination rates.

### Example 2

### Case Studies of Effects of Cholestyramine on SBS

A 51 year old male patient presented with depression, weight loss and lingering flu-like symptoms. It was determined that the patient had a high stress job with frequent travel. The patient further had a long history of symptoms including severe headaches, irritable bowel syndrome, gastroenteritis, and frequent gut-cramps. The patient further presented with numbness in both hands and arms to the elbow. Discussions with the patient revealed that he had worked for may years in “damp and moldy building” with poor ventilation.

The patient was subjected to a VCS screen, which demonstrated heightened VCS, indicating that his symptoms were part of a bio-toxin-mediated illness. Confounding variables were eliminated. The patient was placed on a four-doses-a-day regimen of oral cholestyramine at 9 grams per dose. The numbness in the patient's hands was eliminated, his stomach problems were corrected, severe headaches were eliminated, and his energy level was greatly improved.

### Example 3

### Case Studies of Effects of Cholestyramine on Post-Lyme Disease Syndrome (PLDS)

We report here a series of case reports of PLDS patients with chronic symptoms, including neurologic and neurocognitive impairments, following a tick bite in an endemic Lyme area, refractory to antibiotics, shown to have a deficit in VCS, who responded to CSM in essentially the same manner as had other patients with documented chronic, neurotoxin mediated illness. The symptom abatement and VCS response to CSM therapy in PLDS patients support the hypothesis that PLDS may be a chronic, neurotoxin mediated illness. Each of these cases demonstrates the variability of serologic responses and the unreliability of standard Lyme serologic diagnostic tests compared to VCS in documenting the presence of a tick bite related syndrome, including Lyme Disease. These cases demonstrate that VCS scores are a valuable adjunct to serologic testing in the diagnosis of PLDS. We suggest that a complete neurotoxin history be taken and VCS testing be performed in all patients from Lyme endemic areas who possibly have PLDS in an effort to avoid incorrect diagnoses and ineffective therapy of chronic tick bite related symptoms.

Case 1

A 59 year old white male attorney from Maryland presented with the abrupt onset of right sided Bell's palsy, headache, confusion, ataxia, low grade fever and weakness, in association with an ECM rash on his thigh. He remembered being bitten by a small tick 3 days before the onset of symptoms. He had two previous episodes of illness following a tick bite, with a left-sided Bell's palsy in 1998, and ECM in the setting of a flu-like symptoms in 1989. Additional symptoms in this illness included sensitivity to bright light, blurred vision, tearing of his left eye, metallic taste and diarrhea. His Lyme ELISA was negative, as it had been on both prior occasions of tick-related illness. He had 2 IgG bands on Western blot. His VCS score was abnormal compared to expected values. He was treated with IV ROCEPHIN™ for 3 weeks. His Bell's palsy improved, though his other symptoms persisted. 5 days after the IV was discontinued, patient presented with the abrupt onset of weakness in his left upper extremity, difficulty with fine motor use of his left hand, slurred speech, reduced sensation in his left anterior thigh, subtle left eye field cut, and confusion (couldn't knot his tie or drive a car).

The patient was hospitalized at a tertiary care institution in Baltimore, treated with IV ROCEPHIN™ and high dose steroids. Diagnostic studies, including cerebral angiogram, carotid duplex studies, and MRI of brain were non-diagnostic. His symptoms improved, but he had persistent CN VII weakness, confusion, light sensitivity, blurred vision, arthralgia in right ankle, fatigue, muscle aching, tingling in hands, and a pain, “like an icepick” in his right eye.

CSM, in doses FDA labeled for treatment of hypercholesterolemia (9 grams taken 4 times per day), was initiated following informed consent. VCS scores improved over two weeks, exceeding expected values, and symptoms improved, with complete resolution of symptoms and mild VCS deficit in 4 weeks.

Case 2

15 year old white female had the abrupt onset of vertigo, tinnitus, difficulty maintaining sleep, light sensitivity, fatigue, headache and hearing loss 6 weeks after an illness characterized by fever, abdominal pain, headache, joint pains, splenomegaly and fatigue. The patient had previously been well, frequently engaging in outdoor activities. She remembered several tick bites before her illness but denied a rash. Hearing tests showed a severe right-sided sensorineural hearing loss. Laboratory exams, including LP, routine screening blood tests, and Lyme ELISA were normal. The patient did not respond to multiple medications including meclizine, HCTZ, and promethazine. Antidepressants were recommended.

The patient remained ill. She attended school regularly but became moody, with frequent outbursts of anger over seemingly trivial issues. At her initial visit at the Chronic Fatigue Center (CFC) one-year after her initial episode of vertigo, her VCS was abnormal despite near visual acuity of 20/15. In addition to her vertigo, hearing loss, and headache, she was fatigued, sensitive to bright light and mildly short of breath, needing to stop frequently during basketball practices. Her affect was blunted.

Her Lyme IgM Western Blot was positive. Three weeks of doxycycline was prescribed. Three days after beginning antibiotics, she noted worsening of her headache, fatigue and shortness of breath, with an associated fall in VCS. She improved over the next two weeks, with improvement in headache, fatigue and shortness of breath. VCS slowly improved but the hearing loss and vertigo remained. Repeat hearing tests showed no improvement. After 3 weeks of antibiotics, CSM was initiated, at 9 grams taken 4 times a day. The VCS deficit resolved in three weeks, vertigo ceased and hearing improved, though a mild residual sensorineural loss persisted. Her other symptoms, including depressive affect, resolved.

Case 3

A 52 year old white female was referred by an ophthalmologist for an additional opinion on papillitis affecting the left optic nerve for 9 months. Corrected visual acuity was 20/25 in unaffected right eye and 20/200 in the left eye. Additional symptoms included profound fatigue, metallic taste, headache, migratory joint pain without true arthritis, muscle aches, delayed recovery from normal physical activity, mild confusion, ataxia (with frequent falls), sensitivity to bright light in her right eye and an unusual sensation of pain, described as a “lightning bolt that shoots into a large muscle and explodes”. The patient remembered multiple tick bites, nearly on a daily basis, but denied flu-like illness or ECM rash. Her ELISA was negative. Her VCS was markedly abnormal in her right eye, left eye score was unobtainable. Doxycycline was initiated with significant improvement in symptoms and visual acuity noted in 6 days. Visual acuity was still 20/25 in right eye but was now 20/25 in the left eye. Her other symptoms were improved.

Ophthalmologic exam showed marked improvement in papillitis by day 10. Doxycycline was continued for a total of three weeks. Additional Lyme studies, including Western blot and C6 Peptide ELISA assay were negative. VCS did not improve, however. She had ongoing fatigue, muscle aches, blurred vision despite normal visual acuity, headache, metallic taste; joint pain, especially in the MCP and PIP joints, persisted. CSM was prescribed, with resolution of VCS deficit and abatement of symptoms over the next 4 weeks. The patient remained asymptomatic.

Case 4

55 year old white female accountant was treated for depression, headache, Meniere's disease, stress and fibromyalgia for nearly 10 years without sustained response to multiple medical interventions, including HCTZ, meclizine, antidepressants, massage and stress reduction therapy. Evaluations by numerous physicians and ancillary health care providers showed no laboratory abnormalities or physical abnormalities to explain her symptoms of fatigue, muscle ache, headache, vertigo, reduced ability to concentrate, impaired short term memory, reduced ability to perform simple calculations, cough, weakness, shortness of breath, dysesthesia in nonanatomic distribution in hands, AM stiffness of small joints of wrists and feet, and sensitivity to bright light.

Her physical exam was unremarkable. VCS was abnormal, showing the distinctive deficits of the Hudnell Sign. She remembered multiple tick bites but didn't remember a flu-like illness or ECM rash. Three weeks of doxycycline was prescribed, with a significant worsening of fatigue, vertigo, weakness, and muscle aching noted two days later. VCS scores fell significantly. Patient remained on antibiotics for three weeks with mild improvement in all symptoms but resolution in none. VCS score improved back to the pretreatment level. The CSM protocol was prescribed. VCS scores improved steadily over 6 weeks, with resolution of vertigo, headache, confusion, cough, shortness of breath and dysesthesias. Calculation skills returned to normal. Patient still wears sunglasses on bright days and uses NSAID to help her mild, ongoing small joint stiffness, but otherwise has returned to full health.

Case 5

51 year old male car salesman developed Bell's palsy following a tick bite in 1996. He was treated with amoxicillin, prednisone and valciclovir empirically with partial resolution of is right CN VII weakness. The CN VII weakness resolved over the next 12 months but the patient had residual fatigue, shortness of breath, abdominal pain, muscle ache, delayed recovery from normal activity and recurrent pain at right lateral epicondyle, left shoulder, both knees and both feet. All Lyme serologies were normal.

The patient was recalled to the CLDC in 1999, specifically to take the VCS test. The typical deficit associated with chronic carriage of neurotoxins, the Hudnell Sign, was noted. The patient was treated with the standard CSM protocol for 4 weeks with resolution of the VCS deficits and all of his symptoms that had been present for three years. The patient has remained asymptomatic.

Case 6

35 year old female educational assistant developed persistent migratory joint pain following multiple tick bites without subsequent flu-like illness or ECM rash. She was variously diagnosed as having lupus, fibromyalgia or an “undefined connective tissue disease.” A neurologist performed a spinal tap and diagnosed Lyme disease Treatment with IV ROCEPHIN™ for three weeks did not improve her symptoms. She continued to have pain in both knees, neck, small joints of her wrists (difficulty opening jars) and weakness. Two years later, her impairment of short-term memory became evident to the patient and her coworkers. She continued to work, but developed chronic symptoms of severe fatigue, weakness, muscle aches and cramps, a sensation of pain that felt like a “lightning bolt that exploded into large muscle groups”, intermittent tingling in both thumbs and great toes, sensitivity to bright light, reduced ability to assimilate new knowledge, chronic sinus congestion, vertigo, metallic taste, secretory diarrhea and moodiness. She took high doses of NSAID medications without sustained relief.

At her initial office visit, her physical exam was normal, without evidence of inflammatory joint disease. VCS was abnormal, showing the Hudnell sign. She began the CSM protocol, but felt worse at dose 8 of CSM, with a marked reduction in her VCS score noted. She began taking pioglitazone, with resolution of her previously intensified symptoms. At day 10, when she discontinued the pioglitazone, her intensified symptoms returned. She continued CSM and restarted pioglitazone, taking both for an additional 21 days without recurrence of intensification, evidence of hypoglycemia or abnormalities in liver function tests. Her VCS improved and all symptoms began to diminish. At 8 week follow-up, her VCS was normal and all symptoms abated except for mild fatigue (1 on a scale of 1 to 4) and headache. She has remained well.

Case 7

60 year old white female housewife had a 4 month illness following a tick bite characterized by an ECM rash, fatigue, impairment of function and AM stiffness of small joints of both wrists, symmetric swelling and pain in both hands, MCP and PIP joints lasting over one hour in the morning, stiffness of both knees without swelling, weakness, muscle cramps, light sensitivity, red eyes, chronic cough, shortness of breath (non-smoker), tearing, impairment of recent memory, and vertigo. Work-up by attending physician and consultants, including a rheumatologist, gave a working diagnosis of seronegative RA.

Her physical exam was unremarkable. Lab tests showed a Lyme Western blot with 4 IgG bands. Her VCS was abnormal, showing the Hudnell Sign. Treatment with doxycycline, 100 mg. twice a day for three weeks gave mild improvement in fatigue, but muscle aches, light sensitivity, shortness of breath, and cognitive impairment continued. VCS was not improved.

CSM protocol was initiated. Patient had no significant worsening of symptoms at dose 6-10. As two-week follow-up, her energy was improved. She was mentally sharper with no muscle ache and shortness of breath. VCS was improved, but still not normal. SCM protocol was continued two more weeks with resolution of all symptoms and the VCS deficit. She has remained asymptomatic.

Case 8

60-year-old white female schoolteacher developed multiple symptoms following numerous tick bites. She denied an ECM rash or flu like illness. Over several months she subsequently developed, severe fatigue, chronic joint pain, especially in small joints of hands, wrists, ankles and left elbow, impairment of recent memory, difficulty concentrating, reduced ability to find words, sensitivity to bright light, blurred vision, non-productive cough, shortness of breath (5 steps), muscle ache, severe muscle cramps, a sensation of pain in her heel “like an ice pick stuck in it”, sensitivity of her skin to touch and metallic taste. She sought advice from over 20 physicians without benefit. Multiple medications, including NSAID, antidepressants, narcotic and non-narcotic analgesics, were used without benefit. Laboratory studies were normal. Her working diagnoses were fibromyalgia and chronic pain syndrome.

Her VCS score was abnormal at the Chronic Lyme Illness Center, as was the velocity of flow of red cells in the capillaries of the retina, neural rim and lamina cribosa measured by the Heidelberg Retinal Flowmeter. She began the CSM protocol. At one week following, her VCS score was improved, as was the HRF scores. All additional serologic tests including TNF, and C6 Lyme peptide were normal. After three weeks of CSM she noted marked improvement in symptoms. VCS and HRF continued to improve. She continued CSM for a total of 12 weeks with abatement of all symptoms (though her elbow was still sore to touch), normalization of VCS and HRF scores.

Case 9

18-year-old white female had simultaneous ECM rash noted on left thigh, right knee and right calf following bites of small ticks. She had a low grade fever, fatigue, sensitivity to bright light, blurred vision, tearing, arthralgias involving both knees, ankles and wrists, mild muscle aching primarily involving left shoulder, impairment of recent memory, reduced assimilation of new knowledge and daily headaches. Western Blot was negative. She was treated with doxycycline for three weeks with significant worsening of headache and fatigue, with night sweats and muscle weakness noted on days 3-5 of treatment. Her baseline symptoms continued. A diagnosis of fibromyalgia was suggested.

Four months after completion of the antibiotic course, her VCS was abnormal, showing a deficit previously greatest at 6 cycles per degree of visual arc. CSM protocol was initiated, with interval improvement in 3 weeks. CSM was continued with resolution of all symptoms and VCS deficit in 2 more weeks.

Case 10

37 year old white male had numerous tick bites with fatigue, headache and neck pain. He subsequently developed left-sided Bell's Palsy. Lyme Western Blot IgM and IgG studies were both positive. CSF was positive for Lyme antibodies as well. He was treated with two 3-week courses of IV ROCEPHIN™ and 6 months of PO minocycline, followed by 6 months of amoxicillin. His symptoms of fatigue, weakness, muscle ache, shortness of breath, sensitivity to bright light, red eyes, tearing, headache, memory impairment, reduced concentration, confusion, vertigo, diarrhea, small and large joint pain, and a sensation of pain “like a lightning bolt that shoots into a large muscle group and explodes” continued.

His VCS was positive. CSM protocol was initiated, with worsening of his symptoms noted at dose 6-10 of CSM. He improved slowly and required 20 weeks of CSM to reach maximum improvement in VCS and symptoms. He remains asymptomatic.

### Example 4

### Use of Pioglitazone to Prevent Intensification of Persistent Symptoms Following Cholestyramine Treatment of Patients with Post-Lyme Syndrome

Introduction: Since the first description of Lyme disease there have always been subsets of patients identified with persistent symptoms, refractory to antibiotics, referred to as the Post-Lyme Syndrome (PLDS). A prospective study of 51 patients with long-standing PLDS showed significant reduction of symptoms following use of a cholestyramine (CSM) protocol, in patients who demonstrated particular deficits in visual contrast sensitivity (VCS) not attributable to other causes. The symptoms and VCS deficits in the PLDS patients are similar to those demonstrated by patients with other chronic, neurotoxin-mediated illnesses reported previously. Early in the course of CSM treatment, 53% of the 51 PLDS patients experienced a significant intensification of symptoms, not seen in patients with different biotoxin exposures. Pretreatment with pioglitazone, a PPAR agonist, was shown to prevent the intensification associated with CSM use and to reduce plasma levels of TNF alpha in 12 PLDS patients. The multi-site, prospective clinical trial using pioglitazone and CSM in PLDS patients reported here was conducted to confirm benefit of CSM in PLDS patients and to determine if pretreatment with pioglitazone could prevent the intensification. Implications for further study are discussed.

Methods: 241 patients in 2 centers (Pocomoke, Md. and Chico, Calif.), all of whom had long-standing symptoms following a tick bite or exposure to areas where others had tick bites, and who had a clinical and/or laboratory diagnosis of Lyme Disease, refractory to antibiotics, were pretreated with pioglitazone (pio), 45 mg daily for 10 days. On day 6 of pio, CSM treatment was added. An orally administered checklist monitored symptoms; sequential VCS testing was performed. CSM was continued until treatment endpoints of maximum symptom abatement and maximum improvement in VCS scores were both noted. No antibiotics or other therapeutic interventions were administered during the study. Diagnostic parameters used by referring physicians included a history of erythema chronicum migrans (ECM) rash, ELISA assay, Borrelia burgdorferi Western blot, LUAT assay, PCR, CSF antibodies to B. burgdorferi and culture. Patients with a clinical diagnosis of PLDS but without a positive serologic test were included at the discretion of the attending physician.

Results: No patients had adverse effects from pioglitazone, including hypoglycemia or abnormal liver function tests. 5 of 241 patients receiving pioglitazone experienced significant intensification of symptoms, not severe enough to stop treatment. 83.8% of patients had at least 50% reduction of symptoms following use of the CSM protocol, with complete resolution of symptoms in 23%. 3.4% of patients had no improvement. Presence of distinctive VCS deficits were better correlated with subsequent improvement compared to other diagnostic markers.

Conclusion: This study supports the concept that 1) PLDS is a chronic, neurotoxin-mediated illness and 2) antibiotic treatment should be followed by adjuvant treatment with the CSM protocol. The data also suggest that 3) VCS is an effective diagnostic tool that can detect the effects of neurotoxins in PLDS patients. The well-demonstrated benefit of pioglitazone pretreatment suggests 4) that the pro-inflammatory cytokine, TNF, participates in the intensification reaction.

### Example 5

### Use of Atovaquone (MEPRON™) in Patients Coinfected with Babesia microti and Borrelia burgdorferi with Symptoms Refractory to Antibiotics and Cholestyramine

Background

Reports of successful treatment of Babesia microti infections, occurring concomitantly with infection with Borrelia burgdorferi in 25-50% of patients, have shown benefit of short-term use of atovaquone in combination with azithromycin. Studies involving over 325 patients with Lyme Disease who had symptoms refractory to antibiotics (Post Lyme Syndrome, PLDS) have shown benefit from a treatment protocol using pioglitazone and cholestyramine (CSM) (see above), resulting in reduction of symptoms and improvement in visual contrast sensitivity (VCS) scores. These improvements parallel those seen in patients with symptoms following exposure to biotoxin-forming organisms, including dinoflagellates and fungal species. In the PLDS studies, 10% of patients did not respond to the toxin binding protocol. In this group, patients with co-infection with Babesia were disproportionately represented.

Methods

25 patients with serologic evidence of exposure to Babesia microti and Borrelia burgdorferi with persistent, long term symptoms despite courses of antibiotics, including atovaquone and azithromycin and despite use of the pioglitazone-CSM protocol were enrolled in a double-blinded, placebo-controlled, prospective, crossover clinical trial that received FDA and IRB approval. Patients were given either atovaquone or placebo for 3 weeks in combination with cholestyramine and then crossed over to the other arm of the double-blinded trial. Treatment for 6 additional weeks of atovaquone and cholestyramine, provided on an open label basis, followed. Symptoms and VCS scores were recorded.

Results

22 patients completed the trial. 3 dropped out; 1 for non-study related reasons and 2 because of intolerance to CSM. 1 patient stopped treatment at 9 weeks, feeling no improvement. Not all patients provided all interim VCS data, but all provided exit information. 5 patients had complete resolution of their longstanding symptoms. 16 had significant reduction in number and/or severity of symptoms. There were no adverse effects noted other than mild worsening of symptoms experienced by patients during the first few weeks of atovaquone therapy. Patients noticed clinical improvement near the end of the second 3-week course of atovaquone, with continued improvement noted during the third course of atovaquone in combination with CSM.

Discussion

This study supports the concept that some persistent symptoms in Babesia patients with Lyme Disease are related to the slow release over time of a neurotoxin by Babesia organisms. Resolution of symptoms in these patients is not accomplished by short-term use of antibiotics. Neurotoxin binding protocols employing CSM have a significant adjuvant role in treating patients with chronic symptoms in Lyme patients coinfected with Babesia, but atovaquone must be continued in these patients for a prolonged period of time. Additional studies in coinfected patients are indicated as coinfection rates now exceed 10% in endemic areas. Areas of particular interest include the role of pro-inflammatory cytokines in coinfection, possible extravascular sequestration of viable Babesia organisms and the role of the extrachromosomal 35 kb plastid DNA, homologous to DNA of Euglena, in maintaining viable Babesia organisms, despite reportedly curative courses of antibiotics, in symptomatic coinfected patients.

### Example 6

### Chronic Fatigue Syndrome

Introduction: Our data on use of Visual Contrast Sensitivity (VCS) testing to identify patients who satisfy the CFS case definition and who show with evidence of adverse effects of neurotoxins has led to successful treatment with cholestyramine (CSM). These patients' symptoms appeared following exposure to various toxin-forming organisms, including dinoflagellates, bluegreen algae, fungal species, spirochetes and apicomplexans. An additional cohort of CFS patients have symptoms that resolve with CSM treatment, but recur without ongoing CSM use. A final cohort of patients with CFS have had symptoms related to presence of coagulase negative Staphylococci in their noses, with resolution following eradication and recrudescence following reacquisition of bacteria. The cases of four members of a long-studied CFS family are presented for review and illustration of pertinent points, as are histories of 8 CFS patients, all health care professionals, attending a CDC-CFS training meeting.

Methods: Four hundred patient records from the Chronic Fatigue Center, Pocomoke, Md., were found to satisfy the CDC case definition for CFS. Biotoxin exposures, VCS scores, levels of TNF alpha and 1L-1B were correlated with symptoms. These cases included patients with known exogenous exposure to Pfiesteria, ciguatera, and Chattonella (dinoflagellates), Stachybotrys, Aspergillus, and Penicillium (among other fungal exposures), Borrelia and Babesia (tick borne illnesses), Cylindrospermopsis and Microcystis (bluegreen algae), and coagulase negative Staph species. Possible endogenous sources included patients with chronic soft tissue injury and two patients with Charcot-Marie-Tooth disease. Chemical exposure (methylene diphenyl isocyanates (MDI)), vaporized Teflon, permethrin and hyposulfurous acid mixture, also caused symptoms in a subset of patients diagnosed previously with CFS.

Results: Treatment with CSM of patients with exposure, symptoms and VCS deficits successfully reduced or eliminated symptoms in 92% of the cases. Follow-up analysis of symptoms showed three distinctive patterns a) asymptomatic (exogenous exposure), b) sudden relapse (re-exposure) and c) gradual deterioration (endogenous source). Levels of TNF alpha were higher in than 1L-1B in some exposures; 1L-1B was higher than TNF in others.

### Example 7

### α-MSH on CFS Patients

Following completion of informed consent process, patients will be skin tested by intradermal injection (0.01 cc) for sensitivity to αMSH. Patients with local reactions greater than 10 mm in diameter will not be enrolled in the study. Patients will then be given 0.05 cc as intradermal injection and followed for 3 days looking for change in symptoms and VCS scores. If no adverse reactions occur and patients are improved the intradermal dose will be repeated every 3 days until no further improvement is noted in 2 consecutive doses. αMSH and leptin levels will be drawn at this endpoint. Patients will then advance to subcutaneous injections, given every 3 days, beginning with 0.05 cc (5 units). Patients will continue on every 3 days injection until no further improvement in VCS or symptoms occurs. αMSH will be advanced to every other day and then to daily, once an endpoint to response to every other day injection is noted. Once the patient is on daily αMSH injections, the αMSH dose will increase by 5 units every 3^{rd} day until αMSH levels are raised to normal. Leptin levels will be monitored but are not a therapeutic endpoint. Cytokine (TNF and IL-1B) levels will be measured at that time. Symptoms will be recorded as well as adverse events, VCS scores and physical findings. Once the patient is stable, αMSH injections will be held until symptoms (either 50% of prior symptoms, recorded orally or 50% of severity of fatigue and pain, also recorded orally) begin to recrudesce. VCS will be recorded; cytokine levels will be drawn as well as αMSH and leptin levels.

αMSH injections will be reinitiated daily at the last dose associated with clinical improvement, including symptom abatement and stable VCS. αMSH injections will then be with one half the time previously noted to be associated with relapse between injections. The titration of time between doses will be continued in similar fashion until a steady state is reached. Patients will be monitored monthly for 6 months, looking for abnormal lab values with CBC, CRP, comprehensive metabolic profile, αMSH, leptin and cytokines, history and physical exam. Patients will then be followed every 2 months for 6 months and every 3 months for 12 months.

Contrast sensitivity: Contrast sensitivity is tested using a standardized measure, FACT® (Stereo-Optical, Chicago, Ill., a Gerber Coburn company). This noninvasive, rapid, portable, bedside test is reproducibly reliable when used according to a protocol. Prior, well-controlled studies have shown that the mid frequency deficit, greatest at 6 cycles per degree of visual arc, when associated with known exposure and presence of 4 of 8 categories of symptoms, is a statistically validated measure confirming the presence of a neurotoxin.

Study Objectives

Primary Objectives

To determine the efficacy of parenteral replacement of αMSH in patients with CFS, low αMSH and high leptin levels

Secondary Objectives

To evaluate the safety and tolerability of αMSH given intradermally and subcutaneously.

To delineate the time course of αMSH replacement in CFS patients deficient in αMSH

To demonstrate weekly changes in serum levels of cytokines, leptin and VCS during αMSH treatment of CFS

Study Endpoints

Primary Endpoints

Clinical improvement as measured by reduction of symptoms

Corrected visual contrast sensitivity (VCS) score as measured by FACT®

Secondary Endpoints

Safety and tolerability

Correction of abnormalities in levels of leptin, TNF, and IL-1B

Study Design

The study design is a prospective, pilot treatment trial based on a perceived urgency by chronically affected patients with CFS that they might have a reasonable basis to return to an improved quality of life once a demonstrated biochemical abnormality is corrected. There are no treatment guidelines to follow. The cautious approach to parenteral replacement with αMSH was developed by arbitrary criteria. The attempt to identify a time course of durability of benefit is established by trial and error, with gradual increase in dose and dose-interval. The study will permit association of increasing αMSH with expected reduction in leptin and pro-inflammatory cytokine levels and permit attempted correlation with a marker of neurotoxin effect.

Study Population

Subjects regardless of gender may be enrolled into the clinical trial if they are ≧21 years of age and have been diagnosed with Chronic Fatigue Syndrome with associated low levels of αMSH and high levels of leptin. Subjects will have been unsuccessfully treated for CFS with Questran®. Subjects must meet the following entry criteria within 28 days prior to study enrollment.

Study Drugs

Patients will receive parenteral αMSH in an open label manner for the duration of the clinical trial.

Study Protocol

A properly executed informed consent form would be approved by the site's IRB. Following informed consent, subjects will report to the clinic within 28 days, ensuring adequate time for evaluation of the screening tests necessary to determine eligibility for the study prior to study entry. All patients will have a thorough history and physical exam performed, including demography and weight. Assessment of subject eligibility according to inclusion and exclusion criteria will be performed and included in an individual file for each patient.

Baseline (Day 0) (Study Entry) Evaluations

The following procedures will be performed:

Assessment and recording of adverse events (only SAEs related to study participation).

Symptoms questionnaire

Visual contrast sensitivity using FACT

Study drug dispensation

Laboratory evaluations (including a CBC, GGTP, and metabolic profile, as well as baseline TNF, IL-1B, αMSH and leptin)

Serum HCG in women age 10 to 55 (unless deemed unnecessary)

### Example 8

### Use of Visual Contrast Sensitivity and Cholestyramine in Diagnosis and Treatment of Indoor Air Acquired, Chronic, Neurotoxin-Mediated Illness

The ability to separate black from gray from white is measured by a neurotoxicological test of contrast sensitivity. As opposed to visual acuity, a measure that reflects 100% contrast between black and white, contrast testing isolates the functioning of neurons of the optic nerve and optic radiation that enable a visual image to include an edge. Previous work by Ginsburg and others has shown that deficits in contrast are associated with abnormalities in visually evoked potentials. A large number of normal patients have been studied, providing a reference distribution of normal contrast sensitivity. Deficits in contrast may be permanent, resulting from ophthalmologic diseases, neurodegenerative diseases, and occupational exposure to known neurotoxic substances, including organic solvents, heavy metals and petroleum products. There are no data showing that resident indoor fungal organisms make a neurotoxin(s), but the preliminary study suggests that Stachybotrys may not be the only fungus that is neurotoxigenic and pathogenic.

Contrast sensitivity is tested using a standardized measure, FACT® (Stereo-Optical, Chicago, Ill., a Gerber Coburn company). This noninvasive, rapid, portable, bedside test is reproducibly reliable when used according to a protocol. Prior, well-controlled studies have shown that the mid frequency deficit, greatest at 6 cycles per degree of visual arc, when associated with known exposure and presence of 4 of 8 categories of symptoms, is a statistically validated measure confirming the presence of a neurotoxin.

The mechanism of development of the deficit in the FACT likely is due to local hypoperfusion of neurons of the optic nerve at the capillary level. Until recently, there has been no optical mechanism to demonstrate localized hypoperfusion. A new FDA licensed device, the Heidelberg retinal flow meter (HRF) can show the deficit in perfusion in affected patients, not seen in control patients.

Treatment of indoor air acquired illness (IAAI), variously termed sick building syndrome and building related illness, with antibiotics, removal from the affected environment, asthma and allergy medications does not clear the clinical syndrome in a significant number of patients. Adjuvant therapy with cholestyramine (CSM) in patients with IAAI and symptoms refractory to standard medications and modalities who have a specific deficit in contrast sensitivity (an indicator of the presence of a biological neurotoxin) results in significant improvement in many, but not all of these patients. In the pilot study of Stachybotrys exposed patients, CSM treatment resulted in prompt clinical improvement, with resolution both of symptoms and the deficit on standardized contrast sensitivity testing.

Toxin binding therapy with CSM, at doses FDA labeled for treatment of hypercholesterolemia (9 grams of powder taken 4 times per day), results in a measurable improvement in FACT scores that matches symptomatic improvement in patients in all of the diseases mentioned above. The mechanism of benefit is thought to be an electrostatic interaction of the quaternary ammonium side chains of CSM with a molecular dipole (ion sink) created by the molecular structure of the toxins. The electrostatic interaction holds the toxins in the gut, preventing enterohepatic recirculation, much as CSM binds cholesterol. Elevated cholesterol levels do not cause a deficit in FACT; treatment of high cholesterol patients who aren't affected by toxins does not result in an improvement in FACT. In prior studies, improvement in symptoms and FACT scores was durable with no relapse in the absence of re-exposure. Re-exposure can result in reacquisition of symptoms and FACT deficit that respond to repeat CSM therapy.

This study is intended to show that patients with undetermined exposure to indoor air environments with potential residence of toxin forming fungal species or other unknown sources of a chronic, neurotoxin mediated illness can be detected by a screening process, validated by sophisticated neuro-ophthalmological technology, treated with a benign, non-absorbable anion binding resin, with the benefit of treatment measured by improvement in symptoms, VCS, and retinal or neural rim flow rates. The double-blinded protocol design will provide statistical certainty of benefit in patients.

According to a recent OSHA report, as many as 15% of our work sites and 10% of our schools are potentially sick buildings. Both are increasing in frequency. There are no FDA labeled medication protocols available for treatment of IAAI. By identifying an inexpensive biomarker and providing inexpensive, reliable therapy for IAAI patients, their health will improve. Perhaps of greater importance is the ability to define vague syndromes like SBS and identify the true nature of the illness as a chronic, neurotoxin-mediated illness. The FACT and HRF scores will become validated clinical tools for use in these conditions. The use of a standardized symptom-recording instrument will help clinicians separate chronic, neurotoxin-mediated illness from other conditions, such as depression, stress, allergy, deconditioning, irritable bowel syndrome and fibromyalgia, for example.

An additional benefit for patients will be return to normal health. A benefit for clinicians will be a standardized approach to this challenging clinical problem in which older methods of laboratory testing are unreliable, expensive and controversial. By employing a physiological test and not a serological test, the clinician can document the effect of a pathogenic organism independent of the limited ability to detect the presence of the organism. Once the FACT and HRF are validated as useful screening tools, then clinicians in endemic areas can begin to detect a cohort of patients who have acquired a subclinical illness from contaminated indoor air enviroments. Enhanced detection will result in earlier, more effective treatment of the illness.

Study Objectives

Primary Objectives

To determine the efficacy of CSM in the treatment of IAAI, when used in combination with VCS monitoring, in adult subjects with symptoms possibly due to exposure to neurotoxins in indoor air environments.

Secondary Objectives

To evaluate the efficacy of VCS as a screening tool to detect IAAI

To show that change in blood flow in the capillaries of the optic nerve head is a reliable marker for the presence of IAAI.

To determine the efficacy of CSM when taken prophylactically in patients with a prior neurotoxin mediated illness, associated with exposure to an implicated indoor environment, when re-exposed to the suspected indoor environment

Study Endpoints

Primary Endpoints

Clinical improvement as measured by symptoms

Corrected visual contrast sensitivity (VCS) score as measured by FACT®

Secondary Endpoints

Safety and tolerability

Corrected capillary perfusion rate of the optic nerve head as measured through optic nerve imaging using a Heidelberg retinal flow meter

Failure to develop a recrudescent illness when taking CSM, despite re-exposure with a history of illness acquisition previously associated with exposure when not taking CSM

Study Design

The study design is based on observed clinical benefit in less than 14 days in patients exposed to toxin-forming indoor fungal species, including Stachybotrys. By using 21 days of therapy, this study will likely reproduce previously observed benefit, but with an additional week of treatment to ensure benefit. By providing up to six additional weeks of therapy in the open label phase of treatment, our goal of resolution of symptoms has a better chance to analyze additional possibly confounding factors.

This is a single-center, randomized, double-blinded, placebo-controlled study. The initial phase of the study will use symptom lists and prior environmental and chemical exposure lists to identify patterns of illness in users of the indoor environments. All patients will be screened with VCS. Patients with deficits will then be analyzed for symptoms and confounding environmental factors.

Patients with VCS deficits, presence of symptoms from 4 of 8 categories of neurotoxic symptoms and no confounding exposures will be enrolled in the initial arm of the trial. CSM or placebo will be provided in a double-blinded manner, with randomization done by the staff of the study coordinator, for 21 days. Symptoms and VCS scores will be recorded for both groups at completion of the 21 day efficacy study. Patients will be crossed-over to the other treatment arm for an additional 21 days. Symptoms and VCS will be recorded. The double blind will be broken at this stage. If patients have persistent symptoms or VCS deficits, they will be offered CSM or placebo (at the patients request, depending on which blinded medication helped them, in their opinion, more than the other), provided in an open-label fashion. The second arm of the study will enroll successfully treated patients with normal VCS. They will return to their indoor environment, but without CSM. Possible confounding exposures will be recorded daily. VCS will be recorded daily and symptoms recorded every two days. At the end of 3 weeks of exposure, all patients will be analyzed for possible relapse. Those patients who relapse, with deficits in VCS and symptoms will be retreated with CSM for 21 days or until VCS and symptoms are again corrected. The third arm of the trial will involve patients with re-exposure who relapsed. Once asymptomatic, or at the end of the 21 day CSM treatment, but before returning to the indoor environment, these patients will be treated with a prophylactic dose of CSM, one scoop taken twice a day. These patients will again monitor their VCS scores and symptoms. These patients will be evaluated 3 weeks later. Patients who successfully avoided reacquisition of their previously known, environmentally acquired illness will be provided the opportunity to continue CSM or not. These patients will be allowed to enroll in a long-term follow-up study that is the fourth arm of this trial which attempts to confirm long term prevention of relapse, despite re-exposure. These patients will have VCS and symptom recording monthly. This arm of the trial will continue for 6 months.

HRF measurements will be performed at entry into the study, completion of the 21 days of both placebo and CSM therapy, following 21 days of re-exposure, following 21 days of re-treatment and 21 days of the prevention arm. The HRF is much less portable than VCS and cannot be used for daily measurements on all patients.

Planned Sample Size

A total of 50 subjects at one study site will be enrolled into the study. The sample size was based on experience gathered in previous studies rather than statistical considerations. However, the number of subjects is expected to provide adequate information to evaluate the study objectives.

Study Population

Subjects regardless of gender may be enrolled into the clinical trial if they are ≧18 years of age and have been diagnosed with IAAI. Subjects must meet the following entry criteria within 28 days prior to study enrollment.

Inclusion Criteria

A subject will be eligible for inclusion in this study if all of the following criteria apply:

1. Male or female, at least 18 years of age.

2. History of indoor air exposure.

3. No other known causes of VCS deficits

4. Presence of 4 of 8 symptom categories of neurotoxin illness.

5. Failure to respond to standard medications, including antibiotics, lung and allergy medications.

6. If female, documentation of non- pregnant status.

7. Abnormal visual contrast sensitivity score

8. Corrected visual acuity of 20/50 or better in each eye

9. A signed and dated written informed consent is obtained for the subject or the subject's legally acceptable representative prior to study participation.

Exclusion Criteria

A subject will not be eligible for inclusion in this study if any of the following criteria apply:

1. Participation in other investigational drug protocols within the last 28 days.

2. History of chronic solvent exposure, chronic alcoholism, occupational exposure to metal fumes, dust or petroleum products.

3. Diagnosed with complete biliary obstruction.

Study Drugs

Subjects will be randomized to receive either CSM or placebo, double blind, in combination with Questran®, open-label, for the balance of the study.

Questran® Light

Questran® Light (cholestyramine) is a non-absorbable anion binding resin FDA approved for the treatment of hypercholesterolemia. It is available in powder form for oral suspension. Each 5 grams of Questran Light® contain 4 grams of anhydrous cholestyramine resin and the inactive ingredients: aspartame, citric acid, flavor, color, propylene glycol alginate, colloidal silicon dioxide and sucrose, and xanthan gum. Cholestyramine resin is quite hydrophilic, but insoluble in water, and is not absorbed from the digestive tract. Questran® Light resin absorbs and combines with bile acids in the intestine, of which cholesterol is probably the sole precursor, to form an insoluble complex, which is excreted in the feces.

Questran® Light is contraindicated in patients with complete biliary obstruction where bile is not secreted into the intestine and in those individuals who have shown hypersensitivity to any of its components. Questran® Light may delay or reduce the absorption of concomitant oral medications such as phenylbutazone, warfarin, thiazide diuretics (acidic), or propranolol (basic), as well as tetracycline, penicillin G, phenobarbital, thyroid and thyroxine preparations, estrogens and progestins, and digitalis. Because cholestyramine binds bile acids, Questran® Light may interfere with normal fat digestion and absorption and thus may prevent absorption of fat-soluble vitamins such as A, D, E, and K. Since Questran® Light may bind other drugs given concurrently, it is recommended to take other drugs at least one hour before or 4 to 6 hours after Questran® Light to avoid impeding their absorption. The use of Questran® Light in pregnancy or lactation or by women of childbearing age requires that the potential benefits of drug therapy be weighed against the possible hazards to the mother and child. The most common side effects of cholestyramine are constipation, nausea, bloating, and reflux. Less common side effects are abdominal discomfort and/or pain and flatulence.

For additional information, refer to the product information for Questran® Light.

Administration

One scoop of Questran® Light (5 grams) will be mixed in water or juice and swallowed four times a day on an empty stomach, 30-60 minutes before taking other medication or eating food. Questran® Light should not be taken in its dry form. Always mix Questran® Light with water or other fluids before ingesting.

### Example 9

### Diagnosis and Treatment of Sick Building Syndrome

We report clinical data on 21 patients from five buildings with documented fungal contamination. By using a 5 step clinical protocol, we are able to 1) identify patients potentially affected by exposure to resident indoor toxigenic fungal species, 2) demonstrate clinical improvement with treatment with cholestyramine (CSM), according to a published protocol, 3) demonstrate no relapse with exposure to multiple potentially confounding exposures after treatment, but not from the suspect building, 4) document prospectively reacquisition of the complete syndrome of symptoms, VCS deficits and rising leptin within 3 days of re-exposure to the affected building, off CSM, and 5) repeat treatment with CSM showing resolution of the syndrome. These findings suggest that there are multiple readily identified biomarkers that permit a certain basis for screening, monitoring and treatment of SBS. These data further support the genetic basis of susceptibility that underlies the absence of a dose-response relationship in SBS, potentially rendering surveys for mycotoxins and biologically active components in affected buildings of academic, but not clinical, interest. The study design effectively rules out confounding factors, such as second hand tobacco smoke, that have prevented adequate attribution of causation of illness following exposure to contaminated buildings previously.

Methods

21 patients from 5 buildings coming for treatment of chronic health symptoms at the Chronic Fatigue Center (CFC) in Pocomoke, Md., were identified as having occupational exposure to a building with water intrusion, musty smells or visible mold growth. Each remained symptomatic, without improvement following removal from the suspect building for vacations or other reasons. None of the buildings permitted cigarette smoking on site, only one patient was a smoker; one other had exposure to second hand tobacco smoke. None of the patients reported exposures to other known sources of biotoxins; each did not have evidence of water intrusion or fungal growth in their homes. Patients were informed of an IRB approved SBS study; informed consent was obtained to begin a diagnostic and treatment protocol. Patients were excluded from treatment if they had a history of Lyme disease, Possible Estuarine Associated Syndrome (PEAS), ciguatera, Chattonella, Babesia microti, exposure to fresh water lakes with resident Microcystis or Cylindrospermopsis blue-green algae, history of brown recluse spider bites, chronic alcoholism with a history of hepatic encephalopathy, occupational exposure to solvents, metal fumes or hydrocarbons, untreated asthma, prior diagnosis of hypersensitivity pneumonitis, or ongoing neurologic illness; but not for other preexisting medical conditions. No pregnant or nursing women, or patients under age 18 were included. Patient evaluation included a comprehensive medical history, including recording of biotoxin-associated symptoms, physical exam, pulmonary function testing, and VCS testing. Patients from 4 of 5 buildings had comprehensive laboratory testing including CBC, comprehensive metabolic profile, leptin, MSH, HLA DR by PCR, ADH and osmolality, androgen levels, and CRP. Tape lift or bulk sampling of suspected mold growing on surfaces in affected buildings was performed; P&K Microbiology (Cherry Hill, N.J.) performed fungal identification. No attempt was made to quantify fungal contamination or measure mycotoxin levels in the affected buildings.

All patients were treated with CSM, 9 grams (one scoop) taken four times daily, according to a published protocol for two weeks. Symptoms, VCS scores and leptin levels were recorded after 2 weeks (AC-1, after first CSM) for all patients. Patients then stopped medication and stayed away from the suspect building for at least 4 days. Symptoms and VCS scores were recorded for all patients (HOC, home off CSM). Patients then re-entered the fungal contaminated workplace building, still not taking CSM. They were evaluated within three days. Symptoms, VCS scores and leptin levels were recorded for all patients (BOC, building off CSM). Patients who stayed in the building were then re-treated (BAC-2, building after second CSM) for 2 weeks, with symptoms, VCS and leptin levels recorded. Symptoms for all 5 exposures and VCS scores for all 5 exposures were recorded and compared to published control groups (RES-REC ref).

Vision Tests & Analyses

All subjects who normally wore corrective lenses for near-point viewing were asked to wear them during vision testing. The visual acuity and VCS tests were administered monocularly to each eye; an eye occluder was held over one eye while the other eye was tested. All vision tests were administered under illumination from a “daylight” illuminator (fluorescent source with a correlated color temperature of approximately=6500E K; color rendering index>90; intensity=1150 lux; luminance approximately 70 foot-lamberts) in a clinical unit with normal background lighting. A light meter was used to insure that luminance remained constant throughout the test sessions. A test card holder, consisting of a face rest placed just under the cheek bones or chin as comfort provided, and connected by a calibrated rod to a card holder on the distal end, was used to position the acuity and VCS test cards at a constant distance, previously standardized, from the eyes (acuity—36 cm (14 inches); contrast sensitivity—46 cm (18 inches)).

Near Visual Acuity. The acuity test card (MIS Pocket Vision Guide, ©1997 MIS, Inc.) contained 10 rows of numbers in which the size of the numbers progressed from a larger size in the top row to a smaller size in the bottom row. Participants were asked to first read the numbers in a middle row. Testing proceeded to the next lower row if all numbers were correctly identified or to the next higher row if an error occurred. The Snellen visual acuity of the row (20/20 or 20/30, for example) with the smallest numbers each identified correctly was recorded as the visual acuity score. Two-tailed Student t-tests with an ″=0.05 were performed, using the mean score of each participant's two eyes, to determine if scores differed significantly between cohorts.

Contrast Sensitivity. The contrast sensitivity test card (Functional Acuity Contrast Test, F.A.C.T. 101; Stereo Optical Co., Chicago, Ill., a Gerber-Coburn Co.) contained a matrix (5×9) of circles filled with sinusoidal gratings (dark and light bars). Spatial frequency (1.5, 3, 6, 12 and 18 cycles/degree of visual arc) increased from top to bottom, and contrast decreased from left to right in steps of approximately 0.15 log units. The grating bars were oriented either vertically, or tilted 15 degrees to the left or right. As the investigator called out each circle from left to right, row by row, subjects responded by saying either vertical, left, right or blank. Participants were encouraged to name an orientation if they had any indication that the bars could be seen. Participants were given the option to point in the direction to which the top of the grating was tilted if they felt any difficulty in verbalizing the orientation; none needed this assistance. The contrast sensitivity score for each row (spatial frequency) was recorded as the contrast of the last test patch correctly identified on that row following verification by repeated testing of that patch and the subsequent patch. The procedure was repeated for each row in descending order. The a priori criterion for the inclusion of data in analyses was that the eye has a visual acuity (Snellen Distance Equivalent Score) of 20:50 or better, in order to avoid confounding of the VCS results by excessive optical-refraction error. All eyes met the visual acuity criterion for inclusion in the data analyses.

Data Analysis. The units of analysis for the VCS test were the mean scores of the participant's two eyes at each spatial frequency. The VCS data were analyzed using multivariate analyses of variance (MANOVA, with the Wilks' lambda statistic) procedures suitable for repeated measures with an ″=0.05. The factors in the model were group, spatial frequency, age and their interaction terms. A factor for gender was not included since no gender differences in susceptibility to biotoxin-induced effects had been indicated, and no gender differences in VCS have been reported. Results that showed a significant group-by-spatial frequency interaction were further analyzed in step-down, two-tailed Student t- tests (″=0.05), the equivalent of a univariate ANOVA, to determine which spatial frequencies accounted for the overall effect.

Results

19 patients completed all five arms of the clinical treatment protocol. Two patients did not complete BAC-2, one was asymptomatic (BOC) following remediation of building BPD; the second became completely disabled following her return to her building (BOC) while off medication. She remains in treatment and is not permitted to return to the building. No adverse effects of CSM were reported except for minor nausea, bloating, reflux and constipation. No patients dropped out the protocol because of side effects of CSM.

Patient data from each separate building were combined. Symptoms of the cohort patients are comparable to symptoms of patients with other CBAI previously published or presented. The symptoms and VCS of BASE and BOC are statistically identical. The symptoms and VCS of AC-1, HOC and BAC-2 are statistically identical and are equal to those of known controls (DH).

All patients at baseline testing had evidence of the potential for exposure to toxigenic fungal species; each had at least 4 organ system categories of SBS symptoms present; each had a similar deficit and pattern of deficit of VCS abnormalities. No patients had evidence for potentially confounding exposures to other sources of biotoxins, elements known to cause VCS deficits, or confounding medical conditions. There was no difference in symptoms or VCS scores between smokers and non-smokers (results not shown), though the number of smoker (n=1) and those with second hand smoke (n=2) is too low for analysis. Each patient showed a therapeutic response (AC-1) to the CSM protocol, with return of symptoms and VCS deficits to normal, equaling those of non-exposed controls. Following treatment, exposure to non-building environments (HOC) was not associated with reacquisition of illness. Subsequent exposure to the building was associated with rapid reacquisition of symptoms and VCS deficits (BOC). The re-treatment (BAC-2) cohort showed that patients with SBS can be effectively re-treated in 95% (19/20) of patients. The occurrence of disabling illness in one patient, acquired by working in an affected building for 2 days in a prospective exposure underscores the importance of accurately identifying susceptibility of patients at-risk, before their illness progresses.

Discussion

This study clearly demonstrates the efficacy of a fungal exposure paradigm in diagnosis and treatment of SBS. The value of a comprehensive toxin exposure history, obtained by an experienced physician in a one-on-one interview is confirmed. Self-reported symptom using checklists require little investigator time, but it is our repeated experience that the reported symptoms rarely are confirmed by actual history. Use of a comprehensive questionnaire administered by the investigator, provides documentation of the diversity of symptoms at diagnosis and at each follow-up. The diagnostic information provided by use of VCS as a rapid (<5 minutes), reproducibly reliable, portable, non-invasive, bedside marker of biotoxin effect is also confirmed by these data. The ability of VCS to reflect changes (as opposed to static tests, like antibody levels) within 48-72 hours of change in exposure/treatment provides an additional mandate for its use variously as a screening, diagnostic and follow-up tool. By including the arm that evaluates prospective exposure of a susceptible individual with few symptoms to an affected building in the study, the clinician can confirm epidemiological risk of exposure for reacquisition of SBS. This study design provides strong evidence for the causative role of exposure to toxigenic fungi in genetically susceptible individuals. Previously published epidemiological associations between cases with unknown genotypes and unexposed controls do not provide prospective evaluation of risk. The concept of epidemiologic concept of causation demands an assessment of prospective acquisition of illness.

The role of HLA DR genotypes, analyzed by PCR, as a biomarker of susceptibility supports the concept of genetic predisposition to acquisition of CBAI that precludes the concept of a dose-response relationship in illness acquisition. Illness acquisition will follow exposure, without need for a threshold of exposure for acquisition of symptoms. Higher intensity of exposure or longer exposure will not increase severity of symptoms, VCS scores, or leptin levels in CBAI, as demonstrated in the BOC arm. It is the genetic susceptibility that possibly explains the lack of importance of age, sex, race, duration of exposure, or location of exposure within the building, as factors associated with clinical illness. As an example, patients in two buildings with comparable fungal growth, long-term duration illness, with presumably greater exposure to mycotoxins, such as that of ACC, are no different in symptoms, VCS or leptin than patients with short-term illness, such as BPD, presumably with lesser total exposure to mycotoxins, in symptoms, VCS, or leptin changes. The appearance of 100% disability, refractory to all treatment, in one ACC patient suggests that duration of exposure may cause irreversible effects in a subset of patients of unknown cause. 13/16 affected patients undergoing HLA testing had one or two triplets of HLA DR associated with susceptibility to mycotoxins. The similarity of the genotype (11-7-52B) of the three patients who didn't have a known mycotoxin susceptible genotype to a susceptible genotype (12-7-52B) will require further study; it is likely there is “shading” of HLA DRB1-11 to function similarly to HLA DRB1-12.

HLA DR genotyping is in its infancy with respect to its use as a diagnostic aid in SBS. Prior to PCR technology, only serologic assays of HLA DR were available as clinical tests. These tests did not include as many alleles as PCR and did not permit elucidation of the triplets of genes inherited as links. The appearance of distinct linkages and subsequent susceptibility will need additional verification, as genotypes that are detected as distinct by PCR may be transcribed with only several amino acids differences in the HLA DR product.

The ability of VCS to demonstrate chronic effects in patients without treatment and also to show rapid improvement coincident with treatment makes it a powerful addition to the bedside diagnostic regimen of SBS investigations. The occurrence of the same pattern of deficits of VCS in affected patients, especially with the deficits shown over multiple frequencies, in the absence of optical changes, continues to support the use of VCS as a neurological test. The reproducible appearance of deficits with re-exposure, coincident with recrudescence of symptoms, is consistent with the concept that there is no protective immune response that develops to the antigens contained in extracellular fungal products, including mycotoxins. This finding is consistent with the concept that the HLA DR susceptibility represents defective antigen processing, with resultant “cloaking of the immune response” to fungal toxins as antigens.

The importance of the hypothalamic proopiomelanocortin pathway in toxin-based chronic illnesses, such as SBS, PLDS, and CFS has not been discussed in clinical studies previously. While there is a strong research basis in animal studies on MSH, no studies on elevated leptin levels coincident with MSH deficiency in humans with toxin-based chronic illnesses have not been conducted. The theoretical basis for symptoms of chronic pain and chronic fatigue, in association with MSH deficiency, likely are due to deficiencies of MSH effect on dependent pathways that manufacture endorphins and melatonin, respectively. The mechanism of MSH deficiency via cytokine disruption of the leptin receptor is readily explained by the pro-inflammatory cytokine (PIC) response, especially by IL-1B in SBS patients. PIC readily cross the blood brain barrier and adversely affect the primordial gp 130 cytokine receptor for leptin, blocking initiation of the POMC pathway. In this study, confounding sources of PIC generation have been eliminated. Because of the down-regulating effect of MSH on peripheral PIC production, additional adverse effects on MSH production would occur in MSH deficiency states, further continuing the SBS symptoms. Monitoring effects of PIC from biotoxin exposure/treatment on MSH production is best achieved by monitoring leptin levels, as they change rapidly, whereas MSH changes slowly. Alternatively, monitoring changes in weight, another element related to leptin effects in the hypothalamus, is an indicator of PIC effect on the long form isoform of the leptin receptor.

In this study, the theoretical benefits of measuring leptin and MSH show promise to be of benefit in evaluation and monitoring SBS patients. The wide swings in fasting leptin levels seen in this study likely reflect the hyperacute changes occurring at the level of the long isoform of the leptin receptor in the ventromedial nucleus of the hypothalamus as a result of different cytokine responses to exposure and treatment. We did not measure leptin in the HOC cohort, since symptoms and VCS did not change. Leptin levels changed within 48-72 hours following change in exposure/treatment in several patients in this trial, while MSH changed much more slowly. Additional laboratory biomarkers that change with exposure/treatment currently being studied of potential use in SBS include levels of ADH/osmolality, matrix metalloproteinase-9, plasminogen activator inhibitor-1, cortisol/ACTH, androgens, including androstenedione, DHEA-S, and total testosterone, and vascular epithelial growth factor (VEGF).

### Example 10

### Association of Elevated Levels of Matrix Metalloproteinase 9 with Abnormal Lesions Seen on MRI of Brain in Patients with Exposure to Indoor Toxin-Forming Fungi

Background

Patients with neurologic abnormalities as part of the illness acquired following exposure to indoor toxin-forming fungi (ITFF), leading to “Sick Building Syndrome,” often are found to have lesions seen on MRI scans of the brain. Often called “unidentified bright objects (UBO),” these lesions prompt clinical consideration of demyelinating diseases, including multiple sclerosis (MS). Work-up, including spinal tap, does not confirm MS, yet the symptoms and UBO remain unexplained. Matrix metalloproteinases (MMP) are a large family of collagenase and gelatinase enzymes that are involved with many physiologic processes, including development, tissue remodeling and wound healing; they are associated with pathologic conditions such as spread of metastatic tumors in which extracellular matrix and basement membrane integrity are compromised. MMP have been implicated in the pathogenesis of MS and therapy for MS with interferon-active drugs effectively lowers macrophage release of MMP9. MMP9 can cleave myelin basic protein, releasing encephalitogens associated with development of an autoreactive neuritis. MMP9 levels are also elevated in acute inflammatory arthritis due to Lyme disease (LD) caused by infection with Borrelia burgdorferi (Bb).

We reviewed levels of MMP9 in patients with UBO and exposure to ITFF (n=12) and found significant elevation of MMP9 in serum compared to those ITFF patients without UBO (n=20) and control patients without illness following exposure to ITFF (n=20).

Our work with treatment of the inflammatory mediators of LD, including tumor necrosis factor alpha (TNF), has shown benefit of treatment with a PPAR gamma agonist, pioglitazone, including reduction of TNF and MMP9, especially when combined with a diet that eliminates amylose, with consequent resolution of the inflammatory condition. We hypothesized that elevated levels of MMP9, associated with ITFF and UBOS would be reduced by treatment with pioglitazone and that resolution of neurologic abnormalities and UBOS would accompany definitive treatment of the SBS.

Methods

10 consecutive patients with SBS and UBOS coming for treatment at a private medical clinic were pretreated with pioglitazone before beginning a treatment protocol involving cholestyramine (CSM). Levels of MMP9 were measured before and after pioglitazone and after CSM. MRI scans were done following treatment.

Results

Treatment with pioglitazone resulted in reduction of MMP9 and reduction but not resolution of symptoms. Continuation of therapy with CSM resulted in further reduction of MMP9 and symptoms to levels equal to controls. No significant changes in MRI were seen. There were no complications of therapy.

Conclusions

The complex of symptoms, including neurologic abnormalities, associated with SBS and UBO formation may involve excessive release and/or activation of MMP9. Downregulation of MMP9 release from macrophages, monocytes and keratinocytes by activation of adipocyte PPAR gamma nuclear receptor agonists and reduction of symptoms by use of a mycotoxin binding protocol suggests that a pro-inflammatory cytokine response to putative mycotoxins formed by ITFF is associated with a MS-like clinical syndrome. Further studies, including long-term follow-up with repeated MRI scans, are necessary to evaluate the possible role of MMP9 activation by environmentally acquired biotoxins in de-myelinating disease.

### Example 11

### The Endocrinopathies of Sick Building Syndrome and Deficiency of the Hypothalamic Regulatory Hormone, Alpha Melanocyte Stimulating Hormone: Benefits of Treatment with Cholestyramine

Background

Alpha melanocyte stimulating hormone (MSH) is produced in the proopiomelanocortin pathway (POMC), mainly located in the ventromedial nucleus of the hypothalamus. Among the known functions of MSH are regulation of: 1) melatonin and endorphin production, 2) peripheral pro-inflammatory cytokine release by macrophages, 3) cytokine effects on keratinocyte function in nasal mucosa, 4) pulsatile release of gonadotrophins 5) release of antidiuretic hormone 6) release of ACTH. Deficiencies in MSH are commonly seen in patients with elevated levels of pro-inflammatory cytokines associated with exposure to environmental sources of biotoxin production, including indoor toxin-forming fungi (ITFF), also known as “Sick Building Syndrome (SBS).” Leptin is the agonist of the long isoform of the primordial cytokine receptor that initiates activity of the POMC. An elevated level of leptin is a marker of impaired MSH production.

Many patients with SBS have symptoms suggestive of multiple pituitary and peripheral endocrinopathies, including androgen deficiency, Addison's disease, diabetes insipidus and central obesity. We hypothesized that failure of regulation of anterior and posterior pituitary function due to MSH deficiency was the common element in SBS patients and that treatment of SBS with an orally administered anion binding resin, cholestyramine, would correct the mycotoxin burden of affected patients, with resultant resolution of the endocrinopathies of SBS.

Methods

Following informed consent, 250 consecutive patients with SBS had pre-treatment levels of leptin, MSH, ADH, osmolality, prolactin, DHEAS, androstenedione and testosterone measured; ACTH and cortisol were measured. Because thyroid abnormalities were not found in levels in patients greater than controls, no thyroid measurements were added. Growth hormone was not evaluated. Patients were treated with cholestyramine according to a published protocol. For those patients for whom hormone deficiencies were identified at baseline, post-treatment levels were determined.

Results

MSH deficiency was identified in 92% of patients. Of those, leptin elevation in excess of expected based on BMI was found in 65%, normal leptin in 25% and low leptin in 10%. Those patients with the highest leptin levels had the least improvement in MSH with therapy and the greatest incidence of obesity unresponsive to multiple modalities. Abnormalities in prolactin were uncommon, <5% of patients. Relative ADH deficiency, compared to expected, based on osmolality, was found in 60% of MSH deficient patients. 40% of MSH deficient patients had deficiencies in one or more of the 3 peripheral androgens measured. Deficiency in ACTH, relative to cortisol, was uncommon. Correction of androgen insufficiency was commonly seen in all treated patients; ADH deficiency did not correct quickly and replacement with DDAVP was necessary to correct hyperosmolar states, even in the absence of overt diabetes insipidus. Side effects of ADH replacement were seen in 5% of patients with low dose use of DDAVP. No other significant complications of therapy were noted.

Conclusions

Our expanding understanding of the complex, systemic nature of SBS includes recognition of the effects of excessive pro-inflammatory cytokine responses to environmentally acquired mycotoxins on production of MSH, with consequent development of abnormalities of immune function, mucus membrane function, hypothalamic function, and downstream pituitary function. Deficiency of a hypothalamic neuroendocrine regulatory hormone, MSH, is commonly found in patients with SBS and is associated with reduction of multiple pituitary hormones, with posterior pituitary production of ADH impaired much more than prolactin, and androgen deficiency, presumably due to impaired pulsatile secretion of gonadotrophins, found more frequently than abnormalities in cortisol production. Complete evaluation of affected SBS patients must include measurement of multiple hormones. The role of leptin elevation as a marker of cytokine effects on MSH production must also be recognized as having its own set of complications, including refractory obesity.

### Example 12

### Sick Building Syndrome in Water Damaged Buildings: Generalization of the Chronic Biotoxin-Associated Illness Paradigm to Indoor Toxigenic-Fungi Exposure

This study reports 103 chronically ill patients exposed to 43 “Sick Buildings” who meet all criteria for SBS. All buildings had evidence of water damage; fungi were detected in each building by laboratory analysis, visual inspection and/or musty odor. Cases had a mean of 10.1 symptoms and >60% deficit in VCS before treatment, whereas non-cases averaged 1.2 symptoms and VCS at levels seen in previous control groups. There were no consistent abnormalities in routine laboratory measures, physical exam, and pulmonary function testing. Following CSM therapy, symptoms (1.3) and VCS equaled non-cases and historic controls. Statistically significant differences in symptom number, VCS, and response to CSM were not observed between fungal genera, number of genera, residential and occupational exposure, exposure duration, age, gender, ethnicity or method of fungi detection. Re-exposure following illness resolution resulted in reacquisition of illness, unless CSM was taken prophylactically. Relapse was not seen in the absence of re-exposure.

Subsequent analyses identified 4 genotypes of HLA DR in >90% of cases, not found in exposed but asymptomatic individuals. Deficiencies of melanocyte stimulating hormone (MSH) was observed in 90% of cases, but in <5% of non-cases and controls. Leptin level was elevated in 70% of MSH deficient patients.

I claim:

1. A method of treating one or more of sick building syndrome (SBS), post-Lyme Disease Syndrome (PLDS), and chronic fatigue syndrome (CFS) by administering to a patient in need thereof an amount effective of cholestyramine and/or α-melanocyte stimulating hormone to treat the one or more syndromes.

2. The method of claim 1, comprising administering to the patient an effective amount of cholestyramine.

3. The method of claim 1, comprising administering to the patient an effective amount of α-melanocyte stimulating hormone.

4. The method of claim 1, comprising administering to the patient an effective amount of both cholestyramine and α-melanocyte stimulating hormone.

5. The method of claim 1, wherein the patient suffers from a deficit in visual contrast sensitivity.

6. The method of claim 1 wherein the patient is refractory to standard therapies for the disorder being treated.

7. The method of claim 1 wherein the patient has been exposed to a toxin-forming organism selected from the group consisting of dinoflagellates, fungi, sphirochetes, protozoa, arachnids, and cyanobacteria.

8. The method of claim 1 wherein the patient possesses an HLA genotype comprising one or more allelic triplet selected from the group consisting of (a) DRB1-4, DQ-8, DRB4-53; (b) DRB1-4, DQ7 DRB4-53; (c) DRB1-4, DQ8, and DRB4-53; (d) DRB1-17, DQ-2, DRB3-52A; (e) DRB1-7, DQ2, DRB4-53; (f) DRB1-7, DQ9, DRB4-53; (g) DRB1-13, DQ6, DRB3-52A; (h) DRB1-13, DQ6, DRB3-52B; (i) DRB1-13, DQ6, DRB3-52C; (j) DRB1-17, DQ2, DRB3-52A; (k) DRB1-11, DQ-7, DRB3-52B; (k) DRB1-15, DQ-6, DRB4-51; (1) DRB1-16, DQ-5, DRB4-51; (m) DRB1-11, DQ7, DRB3-52B; (n) DRB1-4, DQ3, DRB4-53; and (o) DRB1-14, DQ-5, DRB3-52B.

9. The method of claims 1 further comprising administering to the patient one or more compounds selected from the group consisting of pioglitazone, rosiglitazone and atovaguone.

10. A method of inhibiting one or more of sick building syndrome (SBS), post-Lyme Disease Syndrome (PLDS), and chronic fatigue syndrome (CFS) by administering to a patient previously diagnosed with and treated for SBS, PLDS, or CFS an amount effective of cholestyramine and/or α-melanocyte stimulating hormone to inhibit the one or more of syndromes.

11. The method of claim 10, comprising administering to the patient an effective amount of cholestyramine.

12. The method of claim 10, comprising administering to the patient an effective amount of α-melanocyte stimulating hormone.

13. The method of claim 10, comprising administering to the patient an effective amount of both cholestyramine and α-melanocyte stimulating hormone.

14. The method of claim 10 wherein the patient possesses an HLA genotype comprising one or more allelic triplet selected from the group consisting of (a) DRB1-4, DQ-8, DRB4-53; (b) DRB1-4, DQ7 DRB4-53; (c) DRB1-4, DQ8, and DRB4-53; (d) DRB1-17, DQ-2, DRB3-52A; (e) DRB1-7, DQ2, DRB4-53; (f) DRB1-7, DQ9, DRB4-53; (g) DRB1-13, DQ6, DRB3-52A; (h) DRB1-13, DQ6, DRB3-52B; (i) DRB1-13, DQ6, DRB3-52C; (j) DRB1-17, DQ2, DRB3-52A; (k) DRB1-11, DQ-7, DRB3-52B; (k) DRB1-15, DQ-6, DRB4-51; (1) DRB1-16, DQ-5, DRB4-51; (m) DRB1-11, DQ7, DRB3-52B; (n) DRB1-4, DQ3, DRB4-53; and (o) DRB1-14, DQ-5, DRB3-52B.

15. A method of treating one or more neurotoxin-associated syndrome, comprising administering to a patient suffering from a neurotoxin-associated syndrome an amount of cholestyramine effective to treat the neurotoxin-associated syndrome, wherein the patient possesses an HLA genotype comprising one or more allelic triplet selected from the group consisting of (a) DRB1-4, DQ-8, DRB4-53; (b) DRB1-4, DQ7 DRB4-53; (c) DRB1-4, DQ8, and DRB4-53; (d) DRB1-17, DQ-2, DRB3-52A; (e) DRB1-7, DQ2, DRB4-53; (f) DRB1-7, DQ9, DRB4-53; (g) DRB 1-13, DQ6, DRB3-52A; (h) DRB1-13, DQ6, DRB3-52B; (i) DRB1-13, DQ6, DRB3-52C; (j) DRB1-17, DQ2, DRB3-52A; (k) DRB1-11, DQ-7, DRB3-52B; (k) DRB1-15 , DQ-6, DRB4-51; (1) DRB1-16, DQ-5, DRB4-51; (m) DRB1-11, DQ7, DRB3-52B; (n) DRB1-4, DQ3, DRB4-53; and (o) DRB1-14, DQ-5, DRB3-52B.

16. The method of claim 15 wherein the neurotoxin-associated syndrome is selected from the group of disorders caused by exposure to one or more neurotoxin-forming organisms selected from the group consisting of dinoflagellates, fungi, spirochetes, protozoa, cyanobacteria, gram-positive bacteria, apicomplexans, and arachnids.

17. The method of claim 16 wherein the neurotoxin-forming organism is selected from the group consisting of Pfiesteria, Ciguatera, Chattonella, Stachybotrys, Penicillium, Aspergillus, Cladosporium, Fusarium, Borrelia, Treponema, Leptospira, Denticola; Babesia, Sarcocystis, Plasmodium; Microcystis, Anabaenopsis Cylindrospermopsis; Bacillus, Clostridia, coagulase negative Staphylococcus, and brown recluse spiders.

18. A diagnostic method for identifying patients with SBS, PLDS, CFS, and/or a neurotoxin-associated syndrome that would best benefit by treatment with cholestyramine and/or α-melanocyte stimulating hormone, comprising identifying those SBS, PLDS, CFS, and/or neurotoxin-associated syndrome patients that have a deficit in visual contrast sensitivity, and treating those patients with the deficit in visual contrast sensitivity with cholestyramine and/or α-melanocyte stimulating hormone.

19. A diagnostic method for identifying patients with SBS, PLDS, CFS, and/or a neurotoxin-associated syndrome that would best benefit by treatment with cholestyramine and/or α-melanocyte stimulating hormone, comprising identifying those SBS, PLDS, CFS, and/or neurotoxin-associated syndrome patients that have an HLA genotype comprising one or more allelic triplet selected from the group consisting of (a) DRB1-4, DQ-8, DRB4-53; (b) DRB1-4, DQ7 DRB4-53; (c) DRB1-4, DQ8, and DRB4-53; (d) DRB1-17, DQ-2, DRB3-52A; (e) DRB1-7, DQ2, DRB4-53; (f) DRB1-7, DQ9, DRB4-53; (g) DRB1-13, DQ6, DRB3-52A; (h) DRB1-13, DQ6, DRB3-52B; (i) DRB1-13, DQ6, DRB3-52C; (j) DRB1-17, DQ2, DRB3-52A; (k) DRB1-11, DQ-7, DRB3-52B; (k) DRB1-15, DQ-6, DRB4-51; (l) DRB1-16, DQ-5, DRB4-51; (m) DR1-11, DQ7, DRB3-52B; (n) DRB1-4, DQ3, DRB4-53; and (o) DRB1-14, DQ-5, DRB3-52B, and treating those patients with the deficit in visual contrast sensitivity with cholestyramine and/or α-melanocyte stimulating hormone.
